(19) United States
(12) Patent Application Publication
(10) Pub. No.: US 2008/0220054 A1
(43) Pub. Date: Sep. 11, 2008

(54) MODULATION OF DRUG RELEASE RATE FROM ELECTROSPUN FIBERS

(76) Inventors: V. Prasad Shastri, Nashville, TN (US); Jay C. Sy, Atlanta, GA (US)

Correspondence Address: Ballard Spahr Andrews & Ingersoll, LLP, SUITE 1000, 999 PEACHTREE STREET, ATLANTA, GA, 30309-3915, US

(21) Appl. No.: 11/872,426

(22) Filed: Oct. 15, 2007

Related U.S. Application Data
(60) Provisional application No. 60/829,458, filed on Oct. 13, 2006.

(51) Int. Cl. A61K 9/70; A61K 47/30

(52) U.S. Cl. 424/443; 514/772

(57) ABSTRACT

Disclosed are co-electrospun polymeric fibers comprising polymers comprising pharmaceutically active agents and/or biologically active agents and capable of release at a combined release rate. Also disclosed are processes for preparing polymeric fibers capable of release at a combined release rate. Also disclosed are processes of modulating delivery rate of pharmaceutically active agents and/or biologically active agents. Also disclosed are processes of delivering pharmaceutically active agents and/or biologically active agents. This abstract is intended as a scanning tool for purposes of searching in the particular art and is not intended to be limiting of the present invention.

### CROSS-REFERENCE TO RELATED APPLICATIONS

This application claims the benefit of U.S. Application No. 60/829,458, filed Oct. 13, 2006, which is hereby incorporated herein by reference in its entirety.

### ACKNOWLEDGMENT

This invention was supported in part by Grant R24-AI47739-03 awarded by the National Institutes of Health. The U.S. government may have certain rights in the invention.

### BACKGROUND

Polymeric fiber matrices can find utility in the preparation of a wide variety of medical devices, including textiles and implantable articles. Such polymeric fibers can be prepared by, for example, an electrospinning technique. Further, such polymeric fibers can include various additives, for example therapeutic preparations, for release to a subject in contact with the polymeric fibers. Conventional polymeric fibrous textiles and implantable articles, however, typically fail to provide methods for modulation of drug release rates. That is, conventional impregnated polymeric fibers release an included additive at a rate dependent in part or whole upon the relative solubility characteristics of the additive vis-à-vis the solubility characteristics of the polymer.

Therefore, despite advances in impregnated polymeric textiles and implantable articles current polymer/therapeutic composites generally lack the ability to tailor the release rate of the included additive. Accordingly, there remains a need for improved polymeric fiber design that allows for modulation of additive release rate.

### SUMMARY

In accordance with the purpose(s) of the invention, as embodied and broadly described herein, the invention, in one aspect, relates to a co-electrospun polymeric fiber comprising a first polymer comprising a first agent, wherein the first pharmaceutically active agent or biologically active agent or biologically active agent is capable of release from the first polymer at a first release rate when the first polymer is not co-electrospun; and a second polymer comprising a second pharmaceutically active agent or biologically active agent, wherein the second pharmaceutically active agent or biologically active agent is capable of release from the second polymer at a second release rate when the second polymer is not co-electrospun, wherein the first release rate is greater than the second release rate, wherein the first pharmaceutically active agent or biologically active agent and the second pharmaceutically active agent or biologically active agent are released from the co-electrospun polymeric fibers at a combined release rate between the first release rate and the second release rate.

In a further aspect, the invention relates to a process for preparing a polymeric fiber capable of delivering a pharmaceutically active agent or biologically active agent comprising the steps of providing a first polymer comprising a first pharmaceutically active agent or biologically active agent, wherein the first pharmaceutically active agent or biologically active agent is capable of release from the first polymer at a first release rate when the first polymer is not co-electrospun; providing a second polymer comprising a second pharmaceutically active agent or biologically active agent, wherein the second pharmaceutically active agent or biologically active agent is capable of release from the second polymer at a second release rate when the second polymer is not co-electrospun; and co-electrospinning the first polymer with the second polymer, wherein the first release rate is greater than the second release rate, wherein the first pharmaceutically active agent or biologically active agent and the second pharmaceutically active agent or biologically active agent are capable of release from the co-electrospun polymeric fibers at a combined release rate between the first release rate and the second release rate.

In a further aspect, the invention relates to a process for preparing a polymeric fiber capable of delivering a pharmaceutically active agent comprising the steps of co-electrospinning a first polymer with a second polymer, thereby providing co-electrospun polymeric fibers, and impregnating the electrospun polymeric fibers with a pharmaceutically active agent or a biologically active agent, wherein the pharmaceutically active agent or the biologically active agent is capable of release from the first polymer at a first release rate when the first polymer is not co-electrospun and capable of release from the second polymer at a second release rate when the second polymer is not co-electrospun; wherein the first release rate is greater than the second release rate; wherein the pharmaceutically active agent or the biologically active agent is capable of release from the co-electrospun polymeric fibers at a combined release rate between the first release rate and the second release rate.

Also disclosed are the products of the disclosed processes.

In a further aspect, the invention relates to a process of modulating delivery rate of a pharmaceutically active agent or biologically active agent comprising the steps of providing a first amount of a first polymer comprising a first pharmaceutically active agent or biologically active agent, wherein the first pharmaceutically active agent or biologically active agent is capable of release from the first polymer at a first release rate when the first polymer is not co-electrospun; providing a second amount of a second polymer comprising a second pharmaceutically active agent or biologically active agent, wherein the second pharmaceutically active agent or biologically active agent is capable of release from the second polymer at a second release rate when the second polymer is not co-electrospun; and co-electrospinning the first polymer with the second polymer, thereby providing a co-electrospun polymeric fiber, wherein the first release rate is greater than the second release rate, wherein the first amount and the second amount are selected to provide a combined release rate for the co-electrospun polymeric fiber that is between the first release rate and the second release rate.

In a further aspect, the invention relates to a process of modulating delivery rate of a pharmaceutically active agent or biologically active agent comprising the steps of co-electrospinning a first amount of a first polymer with a second amount of a second polymer, thereby providing co-electrospun polymeric fibers, and impregnating the electrospun polymeric fibers with a pharmaceutically active agent or a biologically active agent, wherein the pharmaceutically active agent or the biologically active agent is capable of release from the first polymer at a first release rate when the first polymer is not co-electrospun and capable of release from the second polymer at a second release rate when the second polymer is not co-electrospun; wherein the first release rate is greater than the second release rate; wherein the first amount and the second amount are selected to provide a combined release rate for the co-electrospun polymeric fiber that is between the first release rate and the second release rate.

In a further aspect, the invention relates to a process of delivering a pharmaceutically active agent or biologically active agent, the method comprising the steps of providing a co-electrospun polymeric fiber comprising a first polymer comprising a first pharmaceutically active agent or biologically active agent, wherein the first pharmaceutically active agent or biologically active agent is capable of release from the first polymer at a first release rate when the first polymer is not co-electrospun; and a second polymer comprising a second pharmaceutically active agent or biologically active agent, wherein the second pharmaceutically active agent or biologically active agent is capable of release from the second polymer at a second release rate when the second polymer is not co-electrospun, wherein the first release rate is greater than the second release rate, wherein the first pharmaceutically active agent or biologically active agent and the second pharmaceutically active agent or biologically active agent are released from the co-electrospun polymeric fibers at a combined release rate between the first release rate and the second release rate; and contacting the co-electrospun polymeric fiber with a subject, thereby delivering the first pharmaceutically active agent or biologically active agent and the second pharmaceutically active agent or biologically active agent at a combined release rate.

### BRIEF DESCRIPTION OF THE FIGURES

The accompanying drawings, which are incorporated in and constitute a part of this specification, illustrate several aspects and together with the description illustrate the disclosed compositions and methods.

FIG. 1 is a graph illustrating poly(lactic acid) (PLA) fiber diameter and morphology as a function of volume fraction of aqueous phase in a water/oil (W/O) emulsion.

FIG. 2 is an electronic microscope image of PLA fibers obtained by spinning from a single-phase system composed of PLA, chloroform, and 1-methyl-2-pyrrolidinone (NMP).

FIG. 3 is an electronic microscope image of PLA fibers obtained by spinning from a W/O emulsion composed of 2.5 v/v % aqueous phase; the porous nature of the fibers is shown in the inset on the bottom left.

FIG. 4 is an electronic microscope image of PLA fibers obtained by spinning from a W/O emulsion composed of 14 v/v % aqueous phase.

FIG. 5 shows a schematic of the electrospinning process.

FIG. 6 shows the effect of water phase in the emulsion on fiber diameter and morphologies of fibers at various compositions.

FIG. 7 shows a proposed mechanism of emulsion stability.

FIG. 8 shows an electrospun fiber diameter versus percent aqueous phase curve.

FIG. 9 shows the effect of increasing aqueous content of the solution on the viscosity of the electrospinning solution; top: varying rotational speed; bottom: constant rotational speed.

FIG. 10 shows the effect of rotation speed on the viscosity of the electrospinning solution

FIG. 11 shows: Upper left, scanning electron micrograph of electrospun PU at 5000×. Upper right, electrospun collagen (40 mg/ml in 1,1,1,3,3,3-hexafluoro 2-propanol) at 5000×. Lower left, cospun collagen and PU fibers at 5000×. Lower right, zoom of cospun collagen and PU fibers at 20,000×.

FIG. 12 shows a normalized optical density vs. percent collagen. Colorimetric comparison of collagen composition of electrospun scaffold. Samples with varying amounts of collagen were stained with Sirius red. Bound dye was solublized in a basic solution and concentration determined spectrophotometrically.

FIG. 13 shows a scanning electron micrograph of aligned polyurethane fibers collected using custom electrospinning apparatus.

FIG. 14 shows (A): sustained release of Doxycyline (Dox) and supramolecular complex of Dox with methylated beta-cyclodextrin (Dox-CD) from PLA fibers, (B) linear regression fit of the linear release portion of the curve, showing that the addition/complexation of CD to the Dox formulation, results in control over release rate (slope: 0.0061 (Dox) versus 0.0027 (Dox-CD)) and an almost 17% reduction in the burst behavior in the earlier phase of the release (0-2 h). The sustained release behavior can be quantified up to 2-days.

FIG. 15 shows sustained release of BSA from polyurethane and poly(L-Lactic acid) fibers. (A) Release of BSA from PU fibers as a function of aqueous load in the fibers during electrospinning. (B) Sustained release of BSA from PU and PLA fibers and tunability of release by co-spinning of PLA and PU (pink line). Notice that release can be achieved for 15 days and beyond.

FIG. 16 shows short-term sustained release of horseradish peroxidase from PU and PLA fibers.

FIG. 17 shows release curves from four doxycycline-loaded meshes (2 PU & 2 PLA with different loads). Fibers were submersed in 1 ml of PBS at 20° C. Drug loading amounts increased with aqueous volume fraction.

FIG. 18 shows Phosphate buffered saline (PBS) and doxycycline was added to the aqueous fraction of the emulsion at different concentrations. Higher PBS concentrations resulted in higher amounts of doxycycline release.

FIG. 19 shows meshes composed of both PLA and PU co-spun fibers released FITC-BSA in an intermediate manner between meshes that were either pure PLA or PU fibers.

FIG. 20 shows representative electron micrographs of nanofiber mesh. PLA fibers (left) and PU fibers (right) had diameters in the 400-200 nm range. (scale bar 6 μm)

FIG. 21 shows the release of doxycycline was slowed by forming supramolecular complexes with cyclodextrin in a 1:1 ratio by mass. The linear region of the pure doxycycline release curve has approximately double the slope as the complexed drug.

FIG. 22 shows FITC-conjugated bovine serum albumin (FITC-BSA) was released at 20° C. PU fibers loaded with a 15% aqueous volume fraction had a similar release profile to 20% aqueous volume loading with lower ultimate concentrations.

FIG. 23 shows TMB assays demonstrated that HRP was released from electrospun fibers in active form. Enzyme activity was most pronounced in the first 3 hours of the experiment with some measurable activity at 24 hours.

### DETAILED DESCRIPTION

Before the present compounds, compositions, articles, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Methods and materials similar or equivalent to those described herein can be used in the practice or testing of the present invention.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

A. Definitions

As used in the specification and the appended claims, the singular forms “a,” “an” and “the” include plural referents unless the context clearly dictates otherwise. Thus, for example, reference to “a component,” “a polymer,” or “an additive” includes mixtures of two or more such components, polymers, or additives, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance may or may not occur, and that the description includes instances where said event or circumstance occurs and instances where it does not.

As used herein, the term “copolymer” refers to a polymer formed from two or more different repeating units (monomer residues). By way of example and without limitation, a copolymer can be an alternating copolymer, a random copolymer, a block copolymer, or a graft copolymer. A copolymer can, in one aspect, be a segmented polymer.

A “residue” of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “segmented polymer” refers to a polymer having two or more chemically different sections of a polymer backbone that provide separate and distinct properties. These two sections may or may not phase separate. A “crystalline” material is one that has ordered domains (i.e., aligned molecules in a closely packed matrix), as evidenced by Differential Scanning Calorimetry, without a mechanical force being applied. A “noncrystalline” material is one that is amorphous at ambient temperature. A “crystallizing” material is one that forms ordered domains without a mechanical force being applied. A “noncrystallizing” material is one that forms amorphous domains and/or glassy domains in the polymer at ambient temperature.

As used herein, the term “biomaterial” refers to a material that is substantially insoluble in body fluids and tissues and that is designed and constructed to be placed in or onto the body or to contact fluid or tissue of the body. Ideally, a biomaterial will not induce undesirable reactions in the body such as blood clotting, tissue death, tumor formation, allergic reaction, foreign body reaction (rejection) or inflammatory reaction; will have the physical properties such as strength, elasticity, permeability and flexibility required to function for the intended purpose; can be purified, fabricated and sterilized easily; and will substantially maintain its physical properties and function during the time that it remains implanted in or in contact with the body. Biomaterials can also include both degradable and nondegradable polymers.

As used herein, a “medical device” can be defined as a device that has surfaces that contact blood or other bodily fluids in the course of their operation, which fluids are subsequently used in patients. This can include, for example, extracorporeal devices for use in surgery such as blood oxygenators, blood pumps, blood sensors, tubing used to carry blood and the like which contact blood which is then returned to the patient. This can also include endoprostheses implanted in blood contact in a human or animal body such as vascular grafts, stents, stent grafts, medical electrical leads, indwelling catheters, heart valves, and the like, that are implanted in blood vessels or in the heart. This can also include devices for temporary intravascular use such as catheters, guide wires, balloons, and the like which are placed into the blood vessels or the heart for purposes of monitoring or repair.

As used herein, the term “subject” means any target of administration. The subject can be an animal, for example, a mammal (e.g., a human, horse, pig, rabbit, dog, sheep, goat, non-human primate, cow, cat, guinea pig, or rodent), a fish, a bird or a reptile or an amphibian. The term does not denote a particular age or sex. Thus, adult and newborn subjects, as well as fetuses, whether male or female, are intended to be covered. In a further example, the subject can be a human. A “patient” refers to a subject afflicted with a disease or disorder. In one aspect, a patient is diagnosed with the disease or disorder. The term “patient” includes human and veterinary subjects.

As used herein, the term “impregnate,” “impregnated,” and “impregnating” refer to the infuse of a first substance, for example a pharmaceutically active agent or a biologically active agent, into the mass of a second substance, for example a polymer. The first substance can be, for example, chemically bonded to the second substance, absorbed within the second substance, or physically adsorbed onto the second substance.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. In various aspects, a preparation can be administered therapeutically; that is, administered to treat an existing disease or condition. In further various aspects, a preparation can be administered diagnostically; that is, administered to diagnose an existing disease or condition. In further various aspects, a preparation can be administered prophylactically; that is, administered for prevention of a disease or condition. In a further aspect, “administering” and “administration” can refer to administration to cells that have been removed from a subject (e.g., human or animal), followed by re-administration of the cells to the same, or a different, subject.

As used herein, the terms “implanting” or “implantation” refer to any method of introducing a medical device, for example a vascular prosthesis, a stent, or a nerve regeneration scaffold, into a subject. Such methods are well known to those skilled in the art and include, but are not limited to, surgical implantation or endoscopic implantation. The term can include both sutured and bound implantation.

As used herein, the term “effective amount” refers to such amount as is capable of performing the function of the compound or property for which an effective amount is expressed. As will be pointed out below, the exact amount required will vary from process to process, depending on recognized variables such as the compounds employed and the processing conditions observed. Thus, it is not typically possible to specify an exact “effective amount.” However, an appropriate effective amount may be determined by one of ordinary skill in the art using only routine experimentation. In various aspects, an amount can be therapeutically effective; that is, effective to treat an existing disease or condition. In further various aspects, a preparation can be prophylactically effective; that is, effective for prevention of a disease or condition.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity may be maintained, for example, by the use of coating materials such as lecithin, by the maintenance of the required particle size in the case of dispersions and by the use of surfactants. These compositions may also contain adjuvants such as preservatives, wetting agents, emulsifying agents, and dispersing agents. Prevention of the action of microorganisms may be ensured by the inclusion of various antibacterial and antifungal agents such as paraben, chlorobutanol, phenol, sorbic acid, and the like. It can also be desirable to include isotonic agents such as sugars, sodium chloride, and the like. Prolonged absorption of the injectable pharmaceutical form may be brought about by the inclusion of agents, such as aluminum monostearate and gelatin, which delay absorption. Injectable depot forms can be made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “pharmaceutically active agent” includes a “drug” or a “vaccine” and means a molecule, group of molecules, complex or substance administered to an organism for diagnostic, therapeutic, preventative medical, or veterinary purposes. This term include externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA), ribonucleic acid (RNA), or mixtures or combinations thereof, including, for example, DNA nanoplexes. Pharmaceutically active agents include the herein disclosed categories and specific examples. It is not intended that the category be limited by the specific examples. Those of ordinary skill in the art will recognize also numerous other compounds that fall within the categories and that are useful according to the invention. Examples include a radiosensitizer, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hydrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

As used herein, the terms “biologically active agent” and “bioactive agent” mean an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, hormones, antibodies, or therapeutic proteins. Other bioactive agents include prodrugs, which are agents that are not biologically active when administered but, upon administration to a subject are converted to bioactive agents through metabolism or some other mechanism. Additionally, any of the compositions of the invention can contain combinations of two or more bioactive agents.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific aspect or combination of aspects of the disclosed methods.

It is understood that the compositions disclosed herein have certain functions. Disclosed herein are certain structural requirements for performing the disclosed functions, and it is understood that there are a variety of structures which can perform the same function which are related to the disclosed structures, and that these structures will typically achieve the same result.

B. Electrospinning

The technique of electrospinning, also known within the fiber forming industry as electrostatic spinning, of liquids and/or solutions capable of forming fibers, is well known and has been described in a number of patents as well as in the general literature.

Typically, the process of electrospinning generally involves the creation of an electrical field at the surface of a liquid. Fibers produced by this process have been used in a wide variety of applications, and are known, from U.S. Pat. Nos. 4,043,331 and 4,878,908, to be particularly useful in forming non-woven structures. The resulting electrical forces create a jet of liquid which carries electrical charge. Thus, the liquid jets maybe attracted to other electrically charged objects at a suitable electrical potential. As the jet of liquid elongates and travels, it will harden and dry. The hardening and drying of the elongated jet of liquid may be caused by cooling of the liquid, i.e., where the liquid is normally a solid at room temperature; evaporation of a solvent, e.g., by dehydration, (physically induced hardening); or by a curing mechanism (chemically induced hardening). The produced fibers are collected on a suitably located, oppositely charged receiver and subsequently removed from it as needed, or directly applied to an oppositely charged generalized target area.

In one aspect, electrospinning (ES) is an atomization process of fluid which exploits the interactions between an electrostatic field and the fluid. In one aspect, the fluid can be a conducting fluid. During electrospinning, fibers with micron or sub-micron sized diameters are extruded be means of an electrostatic potential from a polymer solution (see U.S. Pat. No. 1,975,504 to Formhals). When an external electrostatic field is applied to a fluid (e.g., a semi-dilute polymer solution or a polymer melt), a suspended conical droplet is in equilibrium with the electric field. Electrostatic atomization occurs when the electrostatic field is strong enough to overcome the surface tension of the liquid. The liquid droplet then becomes unstable and a tiny jet is ejected from the surface of the droplet. As it reaches a grounded target, the material can be collected as an interconnected web containing relatively fine, i.e. small diameter, fibers. The resulting films (or membranes) from these small diameter fibers have very large surface area to volume ratios and small pore sizes. This process typically yields non-woven mats or felts composed of round fibers that are extremely pliable. Due to their high-surface area and good mechanical characteristics, electrospun meshes have traditionally found applications in filtration and composite reinforcement. For the very same reasons, felts and meshes derived from biocompatible polymers such as poly(lactic acid) and its copolymer with glycolic acid and other polyesters are being explored as substrates (scaffolds) for association of cells in the engineering of tissue (see Kenawy et al., Biomaterials, 2003, 24 (6), 907 describing making a fiber by electrospinning process from a single-phase system containing ethylene vinyl alcohol, 70% propanol and 30% water). Such pliable porous media is particularly suited for engineering of skin, vascular, and neural prostheses.

Parameters that can be varied in the ES process are the electric field, the distance between the “Taylor Cone” and the target, and the polymer solution viscosity (Fridrikh et al., G. C. Phys Rev Lett. 2003, 90(14), 144502). Due to the complexity of the fiber forming process, very few attempts have been made to alter geometry of electrospun fibers. Recently, Reneker and coworkers have observed the formation of branched and ribbon-like fibers in some solvent systems and have attributed this to the collapse of a polymer skin due to buckling instability similar to that seen in garden hoses (see Koombhongse et al., Polym. Sci.: Part B: Polym. Phys. 2001, 39, 2598-2606). However, the formation of such fibers is not achievable in a predictable manner under generally known ES operating conditions. U.S. Pat. Nos. 4,323,525 and 4,689,186 to Bornat, incorporated by reference herein, are directed to processes for the production of tubular products by electrostatically spinning a liquid containing a fiber-forming material.

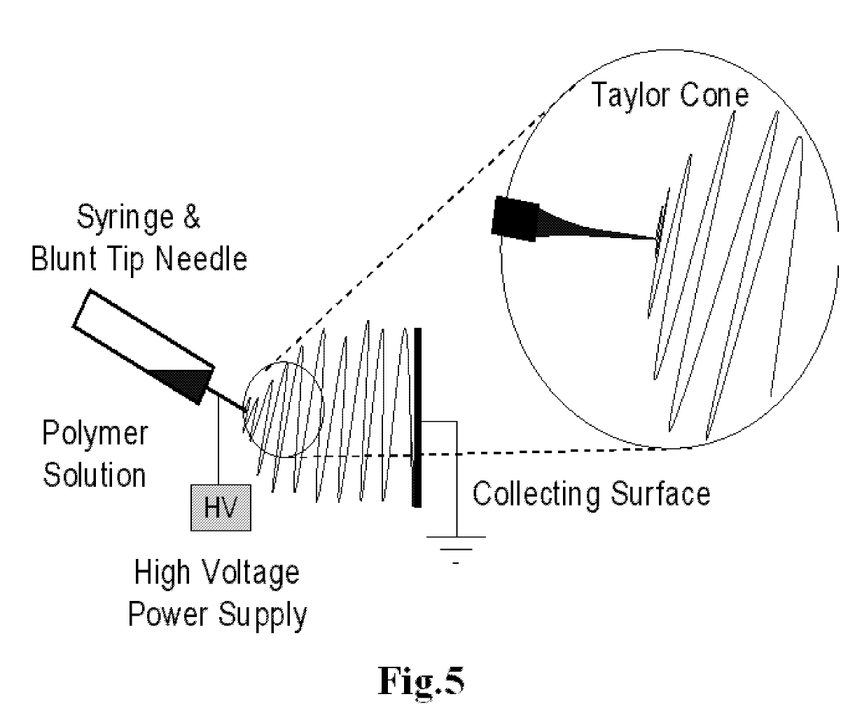

ES is a process through which fibers with micron or sub-micron sized diameters are extruded from a polymer solution by means of an electrostatic potential (FIG. 5). In a typical ES process, the polymer solution is injected through a nozzle while being subjected to a high voltage DC field (e.g., 5-30 kV). Under such conditions, the polymer solution erupts into a “Taylor Cone” due to the droplet being subjected to a phenomenon called “Raleigh's Instability,” which leads to whipping of the polymer jet. As the jet is propelled, the formation of fibers is facilitated by solvent evaporation and thinning of the jet. The parameters that can be varied to affect fiber morphology include the electric field strength, the distance between the “Taylor Cone” and the target and viscosity of polymer solution. Due to the complexity of the “Taylor Cone” formation, most attempts at controlling fiber morphology have focused on controlling polymer solution properties. This can be achieved by either increasing the polymer concentration or molecular weight or increasing volatility of the organic solvent; all of which accelerate the rate at which the polymer fibers solidifies during spinning. In general, increasing viscosity and solvent volatility results in thicker fibers.

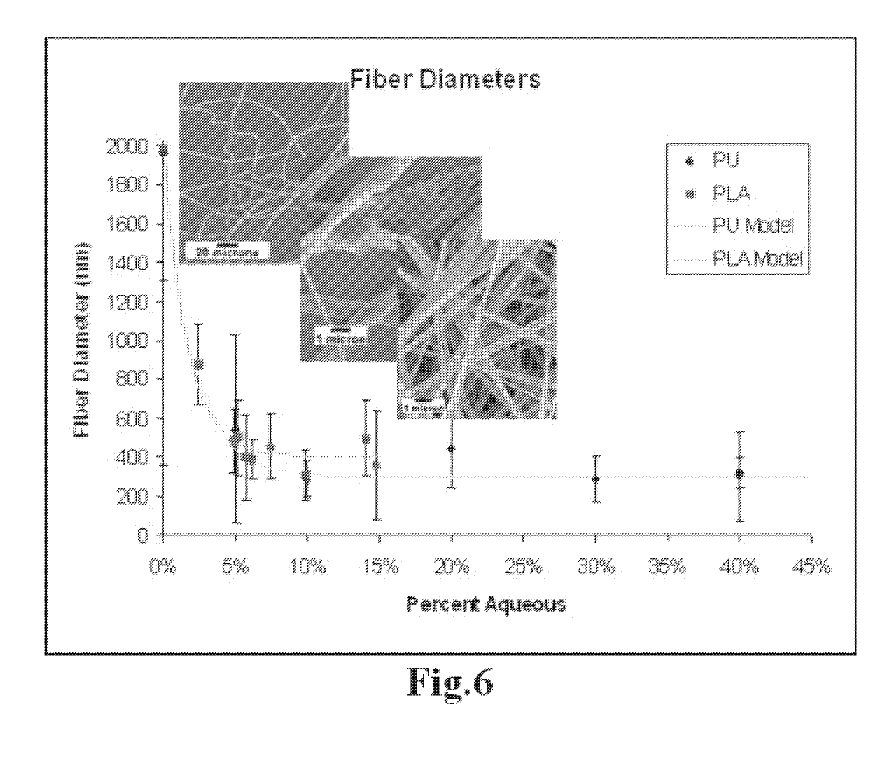

A limitation of conventional approaches is that they do not enable altering of other fiber properties such as aspect ratio (rounder versus flatter fibers) and fiber porosity both of which can severely impact cellular interactions by increasing surface area, which can be a desired property in cell contacting applications and tissue engineering (TE). In contrast, the present invention demonstrates that by using an unstable water/oil emulsion system (i.e., a polymer solution in an organic solvent emulsified with an aqueous phase), the shear thinning behavior of emulsions can be leveraged to spin fibers from polymer solution at low concentration which under normal conditions are not suitable for electrospinning. Using this approach, polymers such as PU and poly(L-lactic acid) have been spun into fibrous mats with fiber diameters ranging from about 10 nm to about 1,000 nm, for example, from about 300 nm to about 2 μm (FIG. 6). Furthermore, in this emulsion-based system, the less volatile water phase has a templating effect on the polymer fiber formation, enabling control over fiber morphology as well. Depending on the modulus of the polymer, fibers ranging from cylindrical, porous to flat-ribbon like can be obtained (FIG. 6).

Conventionally, the diameters of electrospun fibers are achieved by changing polymer concentration or solvent systems. In the disclosed methods, fiber diameter can be controlled without altering the polymer concentration or solvent system. By using an unstable emulsion (water in oil, with small amounts of polymer surfactant), the rheological properties of the multiphase solution are predictably varied, thereby controlling the final polymer fiber dimensions. In addition, using a multiphase solution allows for a templating effect, giving control over fiber porosity and shape. In PLA/chloroform systems, electrospun fibers possess sub-micron diameters (around 400 nm) with ribbon-like and porous morphology at a polymer concentration (2% w/w) that typically yields round fibers with fiber diameters 5-times greater (2 microns). An over two-fold decrease in fiber diameter can be achieved with the addition of just 5% aqueous phase emulsified into the polymer/organic solvent solution. Using emulsions to control fiber shape, diameter, and porosity has desirous applications in many fields including scaffold engineering for vascular, renal, and neural regeneration.

Good mechanical properties, high surface area to weight ratios, and pliability have made electrospun fibers candidates for a wide range of applications in filtration and composite reinforcement. These characteristics, combined with specific polymer properties, also make electrospun felts ideal for tissue engineering scaffolds as well as drug delivery devices. Electrospun materials typically possess a high aspect ratio, which can be a desired property for various application, for example tissue engineering (TE) applications.

Fiber diameter is typically controlled by changing electric field strength (either by changing applied voltage or tip-to-target distance), changing evaporation rates (via changing the spinning environment or using solvents of different volatilities), or by changing polymer concentration. The last method enjoys particular popularity among researchers since polymer concentration is an easy variable to control and can have repeatable and drastic effects on fiber diameters. This method works by changing both the amount of solvent that must evaporate before a solid fiber precipitates from the solution and by changing the viscosity of the solution, and hence, “Taylor cone” formation and final jet diameter.

In conventional methods, surface geometry and morphology of electrospun nanofibers has been more difficult to modify. Typical electrospun fibers adopt a circular cross-section, though porous and flat fiber morphologies have been observed in several polymer/solvent systems, but little research has found success at controlling these morphologies. Common techniques used to modify fiber cross-sectional shape have been to cospin polymers and selectively remove certain polymer phases. More recent approaches have succeeded in producing hollow fiber morphologies by using an immiscible second phase and coaxial spinnerets. Both techniques involve either complicated processing steps or specialized electrospinning apparatus to achieve the desired final shape.

In contrast, the present invention employs a new technique to modulate both fiber morphology and diameter. By emulsifying a second phase into the polymer/volatile solvent solution, the fiber diameter can be decreased by an order of magnitude using a single polymer concentration (in, e.g., the organic phase of the emulsion). Additionally, a range of fiber morphologies ranging from common circular cross-sections, to varying amounts of porosity, to flat, ribbon-like polymer fibers, has been observed with the techniques of the invention. Producing these fibers using the present inventive technique can require neither additional processing steps to selective remove components of the fiber, nor complicated modifications to the traditional electrospinning setup.

One of the most modulated parameters in conventional electrospinning techniques involves changing the concentration of the dissolved polymer. This typically has the effect of being able to control the final fiber diameter by changing how the fiber formation process and timescale during electrospinning. One of the more important parameters coupled with polymer concentration is the viscosity of the solution. Viscosity plays a large role in “Taylor cone” formation and stability.

However, changing the concentration of the polymer solution has two limits. Low concentration solutions can lack the viscosity to properly form a “Taylor cone.” In conventional techniques, instead of drawing a single, electrified jet from the spinneret, the jet is broken down into multiple droplets. This process is called electrospraying and has been utilized in processes like applying surface coatings and inkjet printing. However, the electrospraying process lacks fiber forming properties and results in either a coating of connected droplets or a smooth coating of the dissolved polymer.

At high polymer concentration limits, there are the practical limits of being able to handle such a viscous fluid/gel and feed it to the spinneret. Polymer solutions that are too concentrated are difficult to manipulate and tend to clog the electrospinning apparatus. In addition, extremely high field strengths are required to overcome surface tension to properly form the “Taylor cone.” Such high voltages can be impractical to produce or dangerous. As a result, for practical purposes, most fibers produced by conventional techniques at high polymer concentrations tend to have large diameters that are more easily produced using commercially available techniques.

In one aspect, by adding a second phase to the solution, the methods of the invention artificially increase the viscosity of the spinning solution allowing for the formation of a “Taylor cone” at polymer concentrations that typical electrospray. While not wishing to be bound by theory, the mechanism behind this increase in viscosity is widely believed to be the same mechanism observed in everyday culinary ingredients such as whipped cream and mayonnaise. That is, an increased interaction between the multiple phases can create a higher viscosity than the component parts individually. Multicomponent systems comprising the solvents and polymers of the invention, for example, a polyurethane/chloroform:THF (1:1) system, a poly(1-lactic acid)/chloroform:NMP system, or a poly(ethylene co vinyl acetate)/methylene chloride:NMP system, can be used to provide increased interaction between multiple phases, thereby creating a higher viscosity for the system. In this aspect, it can be possible to spin a polymer solution with a decreased amount of aqueous phase emulsified into the solution.

C. Emulsion-Based Control of Electrospun Fiber Morphology

In a further aspect, fiber morphology can be varied by spinning from a multiphasic fiber-forming medium such as, for example, an emulsion, rather than from a solution or a dispersion. Advantageously, by using at least two-solvent systems having varying evaporation rates and miscibility, morphology of the resulting fiber can be controlled, wherein a preferential evaporation of the more volatile solvent causes the formation of outer surfaces or skins similar to those produced in, for example, a sausage casing process, where the less volatile liquid phase is entrapped and surrounded by a solidified polymer skin. Thus, the invention provides a method for making fibers of different morphologies, including, for example, flattened porous forms. The ability to control morphology of the fiber is useful in various medical applications, such as, for example tissue engineering, drug delivery, as well as non-medical application such as, for example, electronics. Another unexpected benefit of this invention is that due to the addition of aqueous phase, resulting fibers can be produced with small diameters, as compared to the fibers produced from a single-phase solution of identical polymer concentration.

In one aspect, co-spinning, for example co-electrospinning, can be performed by spinning more than one polymer dissolved in a polymer solution, for example a solution of polyurethane and poly(lactic acid). In a further aspect, co-spinning, for example co-electrospinning, can be performed by simultaneously spinning more than one polymer from more than one polymer solution, for example a solution of polyurethane and a solution of poly(lactic acid), using a dual needle system.

Accordingly, the co-electrospinning methods can provides a method of making a fiber from an emulsion comprising a first component including water, and a second component including a polymer dissolved in a solvent. In the method, a force is applied to the emulsion to extrude and separate the emulsion into a fiber. The force is preferably created by an electrostatic field, i.e., an electric force. In this method, the emulsion is preferably electrically conductive or includes electrically conductive materials. Other examples of the force include a magnetic force, an electromagnetic force, or the force of pressurized gas.

Apparatuses useful in this method for creation of the electrostatic field are known in the art such as, for example, electrospinners described by Fridrikh et al. and Bornat (see U.S. Pat. Nos. 4,323,525 and 4,689,186). These apparatuses employ the electric force for spinning the multiphasic fiber-forming medium of the invention. Another type of apparatuses employs a compressed gas as described by U.S. Pat. No. 6,520,425 by Reneker.

The multiphasic fiber-forming medium can be an emulsion, such as, for example, a water/oil emulsion, a double emulsion or an emulsion in which particles are dispersed. In forming the emulsion, at least two components are mixed, wherein the first component (an aqueous phase or a hydrophilic component) has first evaporation rate, and the second component (an oil phase or a lipophilic component) has a second evaporation rate, such that the second evaporation rate is higher than the first evaporation rate.

By varying the ratio of components in the emulsion, desired morphology can be achieved as described herein. In certain aspects, the first component and the second component are provided at a ratio, wherein the ratio is adapted to change morphology of the fiber and its diameter. Examples of fibers with various morphologies include flat fiber, round fiber, porous fiber and combinations thereof. It was observed for an exemplary PLA emulsion, the transition from round to porous fibers occurs in the range of from about 2 to about 5% volume fraction of aqueous phase in the emulsion. Above 5% volume fraction of aqueous phase, fibers with a flat-ribbon morphology are obtained.

In certain aspects, the first component comprises water and optionally, glycerol and poly(vinyl alcohol). In certain aspects, the first component comprises at most 40 vol % of the emulsion. In certain aspects, the first component comprises from about 5 to about 40 vol %, for example, from about 5 to about 20 vol % or from about 5 to about 10 vol %. In certain aspects, the first component comprises 2 to 5 vol %.

In certain aspects, the second component comprises at least 60% of the emulsion. In certain aspects, the second component comprises polymer dissolved in an organic solvent. Non-limiting examples of suitable polymers include poly(styrene), poly(urethane), poly(lactic acid), poly(glycolic acid), poly(ester), poly(alpha-hydroxy acid), poly(ε-caprolactone), poly(dioxanone), poly(orthoester), poly(ether-ester), poly(lactone), poly(carbonate), poly(phosphazane), poly(phosphanate), poly(ether), poly(anhydride), mixtures thereof and copolymers thereof. Further, one or more surfactants, emulsifiers, and/or stabilizers can be added to the emulsion for impacting properties of emulsion such as stability, consistency, etc. Depending on ratios of first component to the second component, the emulsion can be a microemulsion.

In certain aspects, the emulsion can comprise a third component such as for example, a bioactive agent, a pharmaceutically active agent, a cell, a particle, and/or a gel. The third component can be dissolved in either or both of the phases or it can be dispersed. Depending on the choice of the phase, the third component can be located inside or outside of the fiber. For example, if the third component is dissolved in the aqueous phase, upon forming of the fiber, it will be trapped insider, upon evaporation of the solvent of the second phase. Also, if the third component is dissolved in the second phase, upon forming of the fiber, it will be trapped in the outer skin of the fiber. Non-limiting examples of suitable biomolecules include a bioactive polypeptide, a polynucleotide coding for the bioactive polypeptide, a cell regulatory small molecule, a peptide, a protein, an oligonucleotide, a nucleic acid, a poly(saccharide), an adenoviral vector, a gene transfection vector, a drug, and a drug delivering agent. Non-limiting examples of suitable cells include chondroblast, chondrocyte, fibroblast, an endothelial cell, osteoblast, osteocyte, an epithelial cell, an epidermal cell, a mesenchymal cell, a hemopoietic cell, an embryoid body, a stem cell, and dorsal root ganglia. In certain embodiments, the particle is a colloidal particle or a solid particle. Patterning the surfaces of fibers with particles has practical applications, for example, in tissue engineering where presentation of chemical and physical cues on degradable scaffolds allows a more precise control over cell-scaffold interactions. In certain embodiments, the colloidal particle has a diameter of from about 3 nm to about 10 micrometers and includes a polymer, an oxide, a nitride, a carbide, calcium silicate, calcium phosphate, calcium carbonate, a carbonaceous material, a metal, and a semiconductor. In certain embodiments, the solid particle has a diameter of about 3 nm to about 10 micrometers and said solid nanoparticle is a member selected from the group consisting of a polymer, an oxide, a nitride, a carbide, calcium silicate, calcium phosphate, calcium carbonate, a carbonaceous material, a metal, and a semiconductor. An example of incorporation of solid particles is encapsulation silica nanoparticles (SNP) within polymeric fibers. The presence of SNP within the fibers can be verified using SEM and BET measurements, which revealed the presence of a phase with a very high surface area (>50 m²/gm). Also, carbon nanotubes and magnetic particles are examples of solid particles suitable in this invention.

In certain aspects, the particle is a colloidal particle or a solid particle. Patterning the surfaces of fibers with particles has practical applications, for example, in tissue engineering where presentation of chemical and physical cues on degradable scaffolds allows a more precise control over cell-scaffold interactions. In certain aspects, the colloidal particle has a diameter of from about 3 nm to about 10 micrometers and includes a polymer, an oxide, a nitride, a carbide, calcium silicate, calcium phosphate, calcium carbonate, a carbonaceous material, a metal, and a semiconductor.

An example of incorporation of solid particles is encapsulation silica nanoparticles (SNP) within polymeric fibers. The presence of SNP within the fibers was verified using SEM and BET measurements, which revealed the presence of a phase with a very high surface area (>50 m²/gm). Also, carbon nanotubes and magnetic particles are examples of solid particles suitable in this invention.

Non-limiting examples of surfactants include non-ionic surfactants such as, for example, PLURONIC, polyvinyl alcohol, poly(sorbate)(such as, for example, TWEEN-80 and SPAN-200, oleyl alcohol, glycerol ester, sorbitol, carboxy methoxy cellulose or an ionic surfactant such as, for example, sodium dodecyl sulfonate, sodium dodecyl benezene sulfonate, oleic acid, albumin, ova-albumin, lecithin, natural lipids, and synthetic lipids. In certain embodiments, the emulsion comprises water mixed with poly(vinyl alcohol) as the first components and poly(lactic acid) dissolved in organic solvent as the second component, and optionally, silicone oxide nanoparticle having a biomolecule attached to the nanoparticle's surface as the third component.

In certain aspects, the emulsion comprises water mixed with poly(vinyl alcohol) as the first components and poly(lactic acid) dissolved in organic solvent as the second component, and optionally, silicone oxide nanoparticle having a biomolecule attached to the nanoparticle's surface as the third component.

Using a multiphase, emulsified solution in electrospinning affords two controllable fiber characteristics, fiber diameter and surface morphology. This is accomplished by two principles arising from the emulsion system: increase in apparent viscosity and immiscible solvent templating effects.

First, an increase in the apparent viscosity of the solution allows for electrospinning of a lower concentration of the polymer in the compatible solvent. Lower viscosity solutions or solutions with low polymer concentrations tend to electrospray, forming polymer droplets rather than fibers at the grounded electrode. However, by adding additional phases as an emulsion, it is possible to increase the viscosity at the needle tip. This increase in viscosity allows for the formation of a more stable Taylor cone, and thus, produces fiber.

However, increasing polymer concentration does not allow for the formation of ultrafine fibers. It is widely reported that increasing polymer concentration in numerous polymer/solvent systems. However, by using a multiphase solution, the present methods are able to use polymer concentrations that typically electrospray in a one-phase solution. In addition, the methods of invention demonstrate an increase in shear thinning (decrease in viscosity) at the end of the Taylor cone, allowing for even finer fiber formation.

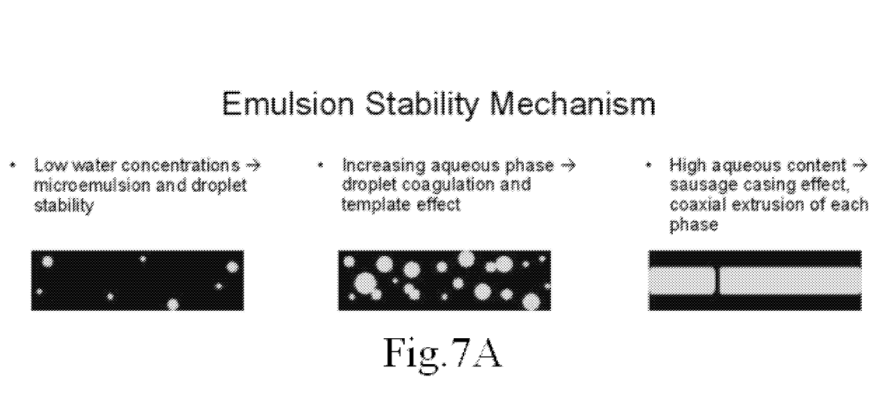

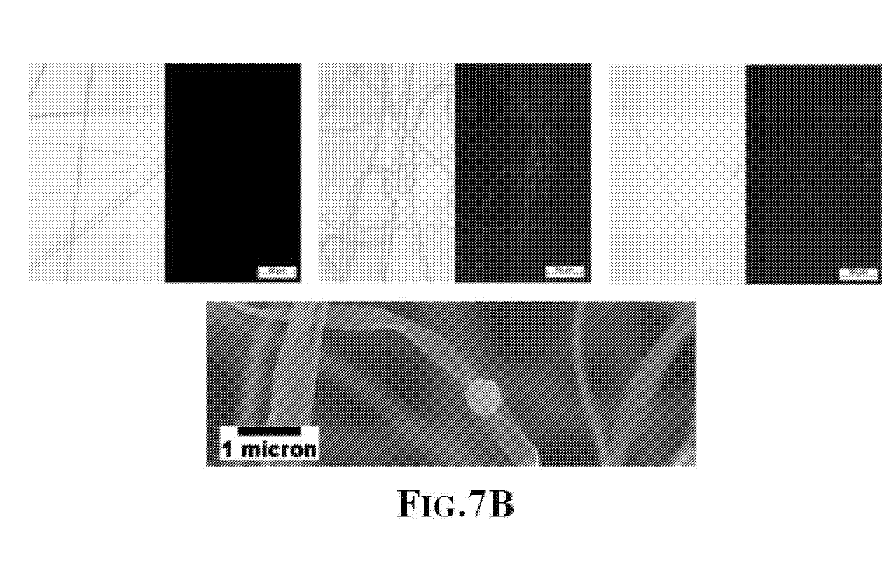

Second, emulsifying a second, immiscible phase into the ES solution allows for templating of the resulting fiber. Researchers have used similar techniques to produce hollow nanofibers by using a coaxial spinneret system. Similarly, the present methods are able to see the transition between solid, round fibers to porous fibers to flat/collapsed hollow fibers. Fiber surface morphology is dependant on the concentration of the immiscible phase. Round fibers are found when very little immiscible phase is emulsified in the polymer/solvent solution. Suspended droplets of the immiscible phase create pores as the concentration of the immiscible phase increases. Eventually, a concentration is reached where droplets coalesce during fiber formation, forming sausage-link-like structures that result in hollow nanotubes. Depending on the modulus/mechanical properties of the polymer of interested, fibers showed either hollow tube morphologies or collapsed, ribbon like structures. In these examples, high molecular weight, high modulus polymers (PLA and Polyox PEO) tend to provide collapsed ribbon structures, while elastomers (PU and PEVA) did not show ribbon-like morphology (see FIG. 7).

By increasing the apparent viscosity of the solution at the spinneret orifice, the present invention can electrospin polymer solutions that typically do not form fibers. As a result, the lower polymer concentrations produce smaller-diameter fibers.

Applications of this co-spinning technique include tethering growth factors to ECM proteins and patterning discrete parts of the scaffold with bioactive signaling molecules, combining different synthetic polymers to more closely match the mechanical properties of native tissue, localizing anti-thrombogenic agents in the graft, and delivering cells to discrete regions of the graft by including them in one of the co-spun fluid phases.

D. Co-Electrospun Polymeric Fibers

In one aspect, the invention relates to a co-electrospun polymeric fiber comprising a first polymer comprising a first pharmaceutically active agent or biologically active agent, wherein the first pharmaceutically active agent or biologically active agent is capable of release from the first polymer at a first release rate when the first polymer is not co-electrospun; and a second polymer comprising a second pharmaceutically active agent or biologically active agent, wherein the second pharmaceutically active agent or biologically active agent is capable of release from the second polymer at a second release rate when the second polymer is not co-electrospun, wherein the first release rate is greater than the second release rate, wherein the first pharmaceutically active agent or biologically active agent and the second pharmaceutically active agent or biologically active agent are released from the co-electrospun polymeric fibers at a combined release rate between the first release rate and the second release rate.

In a further aspect, the first polymer and the second polymer are different polymers or copolymers. In a yet further aspect, the first polymer and the second polymer are the same polymer or copolymer. Each of the polymers can be biodegradable or non-biodegradable. Each of the polymers can be biocompatible. In a still further aspect, one or more of the polymers can be selected from non-biocompatible polymers.

In one aspect, the first pharmaceutically active agent or biologically active agent and the second pharmaceutically active agent or biologically active agent are the same. In a further aspect, the first pharmaceutically active agent or biologically active agent and the second pharmaceutically active agent or biologically active agent can be different.

A polymer can be impregnated with a pharmaceutically active agent and/or a pharmaceutically active agent. That is, for example, a pharmaceutically active agent or biologically active agent can be chemically bonded to the first or second polymer. A pharmaceutically active agent or biologically active agent can be absorbed within the first or second polymer. A pharmaceutically active agent or biologically active agent can be physically adsorbed onto the first polymer.

In a further aspect, the co-electrospun polymeric fiber can further comprise a third polymer comprising a third pharmaceutically active agent or biologically active agent, wherein the third pharmaceutically active agent or biologically active agent is capable of release from the third polymer at a third release rate when the third polymer is not co-electrospun. In a yet further aspect, the third polymer is different from both the first polymer and the second polymer. In a yet further aspect, the third polymer is different from one of the first polymer and the second polymer and the same as the other polymer. In a still further aspect, the third pharmaceutically active agent or biologically active agent is different from both the first pharmaceutically active agent or biologically active agent and the second pharmaceutically active agent or biologically active agent.

1. Concentration

In one aspect, each pharmaceutically active agent or biologically active agent can be, independently, present in or on the polymer in a concentration of from about 0 mg/g to about 500 mg/g, for example, from about 5 mg/g to about 100 mg/g, from about 10 mg/g to about 100 mg/g, from about 50 mg/g to about 500 mg/g, from about 100 mg/g to about 500 mg/g, or from about 100 mg/g to about 300 mg/g. In a further aspect, the pharmaceutically active agent or biologically active agent can be present in or on the polymer in a concentration of from about 0 μg/g to about 500 μg/g, for example, from about 5 μg/g to about 100 μg/g, from about 10 μg/g to about 100 μg/g, from about 50 μg/g to about 500 μg/g, from about 100 μg/g to about 500 μg/g, or from about 100 μg/g to about 300 μg/g. In a still further aspect, the pharmaceutically active agent or biologically active agent can be present in a concentration sufficient to provide and effective amount of the pharmaceutically active agent or biologically active agent when administered to a subject.

2. Pharmaceutically Active Agents

It is understood that, in various aspects, a pharmaceutically active agent can be any pharmaceutically active agent known to those of skill in the art and can be selected to treat or prevent one or more specific diseases or disorders. It is also understood that a pharmaceutically active agent can be selected to stimulate or facilitate a biological process, for example, cell proliferation or bone regrowth. Further, it is understood that a pharmaceutically active agent can be selected based upon its solubility properties vis-à-vis a selected solvent, polymer, or emulsion system.

3. Biologically Active Agents

It is understood that, in various aspects, a biologically active agent can be any biologically active agent known to those of skill in the art and can be selected to treat or prevent one or more specific diseases or disorders. It is also understood that a biologically active agent can be selected to stimulate or facilitate a biological process, for example, cell proliferation or bone regrowth. Further, it is understood that a biologically active agent can be selected based upon its solubility properties vis-à-vis a selected solvent, polymer, or emulsion system.

4. Release Rate

In one aspect, the first release rate can be from about 1 a.u./mg/hr to about 50 a.u./mg/hr, for example, from about 5 a.u./mg/hr to about 50 a.u./mg/hr, from about 5 a.u./mg/hr to about 25 a.u./mg/hr, from about 10 a.u./mg/hr to about 50 a.u./mg/hr, from about 10 a.u./mg/hr to about 25 a.u./mg/hr, from about 5 a.u./mg/hr to about 10 a.u./mg/hr, or from about 5 a.u./mg/hr to about 15 a.u./mg/hr. In a further aspect, the first release rate can be from about 1 μg/mg/hr to about 50 μg/mg/hr, for example, from about 5 μg/mg/hr to about 50 μg/mg/hr, from about 5 μg/mg/hr to about 25 μg/mg/hr, from about 10 μg/mg/hr to about 50 μg/mg/hr, from about 10 μg/mg/hr to about 25 μg/mg/hr, from about 5 μg/mg/hr to about 10 μg/mg/hr, or from about 5 μg/mg/hr to about 15 μg/mg/hr.

In one aspect, the second release rate can be from about 1 a.u./mg/hr to about 50 a.u./mg/hr, for example, from about 5 a.u./mg/hr to about 50 a.u./mg/hr, from about 5 a.u./mg/hr to about 25 a.u./mg/hr, from about 10 a.u./mg/hr to about 50 a.u./mg/hr, from about 10 a.u./mg/hr to about 25 a.u./mg/hr, from about 5 a.u./mg/hr to about 10 a.u./mg/hr, or from about 5 a.u./mg/hr to about 15 a.u./mg/hr. In a further aspect, the second release rate can be from about 1 μg/mg/hr to about 50 μg/mg/hr, for example, from about 5 μg/mg/hr to about 50 μg/mg/hr, from about 5 μg/mg/hr to about 25 μg/mg/hr, from about 10 μg/mg/hr to about 50 μg/mg/hr, from about 10 μg/mg/hr to about 25 μg/mg/hr, from about 5 μg/mg/hr to about 10 μg/mg/hr, or from about 5 μg/mg/hr to about 15 μg/mg/hr.

In one aspect, the third release rate can be from about 1 a.u./mg/hr to about 50 a.u./mg/hr, for example, from about 5 a.u./mg/hr to about 50 a.u./mg/hr, from about 5 a.u./mg/hr to about 25 a.u./mg/hr, from about 10 a.u./mg/hr to about 50 a.u./mg/hr, from about 10 a.u./mg/hr to about 25 a.u./mg/hr, from about 5 a.u./mg/hr to about 10 a.u./mg/hr, or from about 5 a.u./mg/hr to about 15 a.u./mg/hr. In a further aspect, the third release rate can be from about 1 μg/mg/hr to about 50 μg /mg/hr, for example, from about 5 μg/mg/hr to about 50 μg/mg/hr, from about 5 μg/mg/hr to about 25 μg/mg/hr, from about 10 μg/mg/hr to about 50 μg/mg/hr, from about 10 μg/mg/hr to about 25 μg/mg/hr, from about 5 μg/mg/hr to about 10 μg/mg/hr, or from about 5 μg/mg/hr to about 15 μg/mg/hr.

In one aspect, the combined release rate can be from about 1 a.u./mg/hr to about 50 a.u./mg/hr, for example, from about 5 a.u./mg/hr to about 50 a.u./mg/hr, from about 5 a.u./mg/hr to about 25 a.u./mg/hr, from about 10 a.u./mg/hr to about 50 a.u./mg/hr, from about 10 a.u./mg/hr to about 25 a.u./mg/hr, from about 5 a.u./mg/hr to about 10 a.u./mg/hr, or from about 5 a.u./mg/hr to about 15 a.u./mg/hr. In a further aspect, the combined release rate can be from about 1 μg/mg/hr to about 50 μg /mg/hr, for example, from about 5 μg/mg/hr to about 50 μg/mg/hr, from about 5 μg/mg/hr to about 25 μg/mg/hr, from about 10 μg/mg/hr to about 50 μg/mg/hr, from about 10 μg/mg/hr to about 25 μg/mg/hr, from about 5 μg/mg/hr to about 10 μg/mg/hr, or from about 5 μg/mg/hr to about 15 μg/mg/hr.

E. Articles

In one aspect, the invention relates to a bandage comprising the disclosed co-electrospun polymeric fiber. That is, in one aspect, the article can be a nonwoven matting or textile comprising the disclosed polymeric fibers. Similarly, the disclosed methods can be used in connection with a bandage comprising the disclosed co-electrospun polymeric fibers.

In a further aspect, the invention relates to an implantable article comprising the disclosed co-electrospun polymeric fiber. That is, in one aspect, the article can be, for example a polymer disc or chip for anti-tumor or hormone therapy or synthetic bone or cartilage, comprising the disclosed polymeric fibers. Similarly, the disclosed methods can be used in connection with an implantable article comprising the disclosed co-electrospun polymeric fibers.

In a yet further aspect, the invention relates to a synthetic conduit or vascular graft, as disclosed in published U.S. patent application 2006/0085063 for “Nano- and micro-scale engineering of polymeric scaffolds for vascular tissue engineering” to Shastri et al. (incorporated herein by reference in its entirety), comprising the disclosed co-electrospun polymeric fibers. Similarly, the disclosed methods can be used in connection with a synthetic conduit or vascular graft comprising the disclosed co-electrospun polymeric fibers.

F. Processes for Preparing Polymeric Fibers

In one aspect, the invention relates to a process for preparing a polymeric fiber capable of delivering a pharmaceutically active agent or biologically active agent comprising the steps of providing a first polymer comprising a first pharmaceutically active agent or biologically active agent, wherein the first pharmaceutically active agent or biologically active agent is capable of release from the first polymer at a first release rate when the first polymer is not co-electrospun; providing a second polymer comprising a second pharmaceutically active agent or biologically active agent, wherein the second pharmaceutically active agent or biologically active agent is capable of release from the second polymer at a second release rate when the second polymer is not co-electrospun; and co-electrospinning the first polymer with the second polymer, wherein the first release rate is greater than the second release rate, wherein the first pharmaceutically active agent or biologically active agent and the second pharmaceutically active agent or biologically active agent are capable of release from the co-electrospun polymeric fibers at a combined release rate between the first release rate and the second release rate. In a further aspect, the process further comprises the step of providing a third polymer comprising a third pharmaceutically active agent or biologically active agent, wherein the third pharmaceutically active agent or biologically active agent is capable of release from the third polymer at a third release rate when the third polymer is not co-electrospun, wherein the third polymer is co-electrospun with the first polymer and the second polymer.

In a further aspect, the invention relates to a process for preparing a polymeric fiber capable of delivering a pharmaceutically active agent comprising the steps of co-electrospinning a first polymer with a second polymer, thereby providing co-electrospun polymeric fibers, and impregnating the electrospun polymeric fibers with a pharmaceutically active agent or a biologically active agent, wherein the pharmaceutically active agent or the biologically active agent is capable of release from the first polymer at a first release rate when the first polymer is not co-electrospun and capable of release from the second polymer at a second release rate when the second polymer is not co-electrospun; wherein the first release rate is greater than the second release rate; wherein the pharmaceutically active agent or the biologically active agent is capable of release from the co-electrospun polymeric fibers at a combined release rate between the first release rate and the second release rate.

G. Polymers

In one aspect, the polymer fibers can comprise any biocompatible polymer known to those of skill in the art. It is understood that a polymer can be selected based upon its solubility properties vis-à-vis a selected pharmaceutically active agent, biologically active agent, solvent, or emulsion system.

In a further aspect, the polymer fibers comprise poly(lactic acid), poly(glycolic acid), or poly(ε-caprolactone), or a copolymer thereof, or a mixture thereof In a further aspect, the polymer of the fibers can be polyethylene and/or polyurethane. In a further aspect, a polymer can be poly(lactide-co-glycolide), poly(lactic acid), poly(glycolic acid), poly(glaxanone), poly(orthoesters), poly(pyrolic acid), and poly(phosphazenes). Additional polymers that can be used include, but are not limited to, polyalkylene polymers and copolymers, fluorocarbon polymers and copolymers, polyester polymers and copolymers, polyether polymers and copolymers, silicone polymers and copolymers, and polyurethane polymers and copolymers. Other polymers that can be used include, but are not limited to, polyethylenes, polypropylenes, polytetrafluoroethylenes, poly(tetrafluoroethylene-co-hexafluoropropenes), modified ethylene-tetrafluoroethylene copolymers, ethylene chlorotrifluoroethylene copolymers, polyvinylidene fluorides, polyethylene oxides, polyethylene terephthalates, silicones, polyurethanes, polyether block amides, and polyether esters. In a further aspect, the polymer can be one or more polymers, for example, polypyrrole, polyaniline, polythiophene, poly(p-phenylene vinylene), polyparalene, or a mixture thereof. In a further aspect, the polymer can be poly (ethylene-vinyl acetate).

Non-limiting examples of suitable polymers include poly(styrene), poly(urethane), poly(lactic acid), poly(glycolic acid), poly(ester), poly(alpha-hydroxy acid), poly(r-caprolactone), poly(dioxanone), poly(orthoester), poly(ether-ester), poly(lactone), poly(carbonate), poly(phosphazene), poly(phosphanate), poly(ether), poly(anhydride), mixtures thereof and copolymers thereof.

In one aspect, the polymer fibers comprise polyurethane fibers. Such polyurethanes include aliphatic as well as aromatic polyurethanes. In one aspect, useful polyurethanes include aromatic polyether polyurethanes, aliphatic polyether polyurethanes, aromatic polyester polyurethanes, aliphatic polyester polyurethanes, aromatic polycaprolactam polyurethanes, and aliphatic polycaprolactam polyurethanes. In a further aspect, useful polyurethanes include aromatic polyether polyurethanes, aliphatic polyether polyurethanes, aromatic polyester polyurethanes, and aliphatic polyester polyurethanes.

In a further aspect, the polymer fibers comprise segmented polyurethane fibers, for example, a poly(ether-urethane), a poly(ester-urethane), a poly(urea-urethane), a poly(carbonate-urethane), or mixture thereof. In a further aspect, the polymer fibers can be one or more degradable polyurethanes derived from glycerol and sebacic acid. See Wang Y., Ameer G. A., Sheppard B. J., Langer R., A tough biodegradable elastomer, Nature Biotechnology, 2002, 20(6):602-606. In a further aspect, the polymer fibers comprise medical grade and/or FDA-approved polyurethane fibers.

The chemistry of polyurethanes is extensive and well developed. Typically, polyurethanes are made by a process in which a polyisocyanate is reacted with a molecule having at least two hydrogen atoms reactive with the polyisocyanate, such as a polyol. That is, the polyurethane can be the reaction product of the following components: (A) a polyisocyanate having at least two isocyanate (—NCO) functionalities per molecule with (B) at least one isocyanate reactive group, such as a polyol having at least two hydroxy groups or an amine. Suitable polyisocyanates include diisocyanate monomers, and oligomers. The resulting polymer can be further reacted with a chain extender, such as a diol or diamine, for example. The polyol or polyamine can be a polyester, polyether, or polycarbonate polyol, or polyamine, for example.

Polyurethanes can be tailored to produce a range of products from soft and flexible to hard and rigid. They can be extruded, injection molded, compression molded, and solution spun, for example. Thus, polyurethanes can be important biomedical polymers, and are used in implantable devices such as artificial hearts, cardiovascular catheters, pacemaker lead insulation, etc.

In one aspect, the polymer fibers comprise a commercially available polyurethane useable for implantable applications. Commercially available polyurethanes used for implantable applications include ST1882 segmented polyether aromatic polyurethanes available from Stevens Urethane, Easthampton, Mass.; BIOSPAN® segmented polyurethanes available from Polymer Technology Group of Berkeley, Calif.; PELLETHANE® segmented polyurethanes available from Dow Chemical, Midland, Mich.; and TECOFLEX® and TECOFLEX® segmented polyurethanes available from Thermedics, Inc., Woburn, Mass. These polyurethanes and others are described in the article “Biomedical Uses of Polyurethanes,” by Coury et al., in Advances in Urethane Science and Technology, 9, 130-168, eds. K. C. Frisch and D. Klempner, Technomic Publishing Co., Lancaster, Pa. (1984). Typically, polyether polyurethanes exhibit more biostability than polyester polyurethanes, and are therefore generally preferred polymers for use in biological applications.

Polyether polyurethane elastomers, such as PELLETHANE® 2363-80A (P80A) and 2363-55D (P55D), which can be prepared from polytetramethylene ether glycol (PTMEG) and methylene bis(phenyliisocyanate) (MDI) extended with butanediol (BDO), are widely used for implantable cardiac pacing leads. Pacing leads are insulated wires with electrodes that carry stimuli to tissues and biologic signals back to implanted pulse generators. The use of polyether polyurethane elastomers as insulation on such leads has provided significant advantage over silicone rubber, primarily because of the higher tensile strength and elastic modulus of the polyurethanes.

Examples of commercial polyurethanes that can be used in connection with the invention include TECOFLEX®, TECOTHANEL®, and BIOSPAN® polyurethanes. TECOFLEX® segmented polyurethanes are a family of aliphatic, polyether-based thermoplastic polyurethanes (TPUs) available over a wide range of durometers, colors, and radiopacifiers. These resins are generally easy to process and typically do not yellow upon aging. TECOTHANE® segmented polyurethanes are a family of aromatic, polyether-based TPUs available over a wide range of durometers, colors, and radiopacifiers. Generally, TECOTHANE® resins exhibit improved solvent resistance and biostability when compared with TECOFLEX® resins of equal durometer. As with any aromatic polyurethane, TECOTHANE® resins can tend to yellow upon aging or when subjected to radiation sterilization. BIOSPAN® segmented polyurethane (SPU) is a biomaterial widely used in clinical ventricular assist devices and artificial heart cases. It is one of the most extensively tested biomaterials on the market. BIOSPAN® is an elastomeric biomaterial exhibiting a superior combination of physical and mechanical properties together with biological compatibility.

Further examples of commercial polyurethanes that can be used in connection with the invention include Sancure 2710® and/or Avalure UR 445® (which are equivalent copolymers of polypropylene glycol, isophorone diisocyanate, and 2,2-dimethylolpropionic acid, having the International Nomenclature Cosmetic Ingredient name “PPG-17/PPG-34/IPDI/DMPA Copolymer”), Sancure 878®, Sancure 815®, Sancure 1301®, Sancure 2715®, Sancure 1828®, Sancure 2026®, Sancure 1818®, Sancure 853®, Sancure 830®, Sancure 825®, Sancure 776®, Sancure 850®, Sancure 12140®, Sancure 12619®, Sancure 835®, Sancure 843®, Sancure 898®, Sancure 899®, Sancure 1511®, Sancure 1514®, Sancure 1517®, Sancure 1591®, Sancure 2255®, Sancure 2260®, Sancure 2310®, Sancure 2725®, and Sancure 12471® (all of which are commercially available from BFGoodrich, Cleveland, Ohio), Bayhydrol DLN (commercially available from Bayer Corp., McMurray, Pa.), Bayhydrol LS-2033 (Bayer Corp.), Bayhydrol 123 (Bayer Corp.), Bayhydrol PU402A (Bayer Corp.), Bayhydrol 110 (Bayer Corp.), Witcobond W-320 (commercially available from Witco Performance Chemicals), Witcobond W-242 (Witco Performance Chemicals), Witcobond W-160 (Witco Performance Chemicals), Witcobond W-612 (Witco Performance Chemicals), Witcobond W-506 (Witco Performance Chemicals), NeoRez R-600 (a polytetramethylene ether urethane extended with isophorone diamine commercially available from Avecia, formerly Avecia Resins), NeoRez R-940 (Avecia Resins), NeoRez R-960 (Avecia Resins), NeoRez R-962 (Avecia Resins), NeoRez R-966 (Avecia Resins), NeoRez R-967 (Avecia Resins), NeoRez R-972 (Avecia Resins), NeoRez R-9409 (Avecia Resins), NeoRez R-9637 (Avecia), NeoRez R-9649 (Avecia Resins), and NeoRez R-9679 (Avecia Resins).

In a further aspect, the polymer fibers are aliphatic polyether polyurethanes. Examples of such aliphatic polyether polyurethanes include Sancure 2710® and/or Avalure UR 445®, Sancure 878®, NeoRez R-600, NeoRez R-966, NeoRez R-967, and Witcobond W-320.

In the segmented polymers of the invention, the soft segments can be any of those typically used in segmented polyurethanes, such as those disclosed in U.S. Pat. No. 4,873,308 (Coury et al.). The soft segments can include ether groups, ester groups, carbonate groups, urea groups, branched hydrocarbon groups, silicone groups, and the like. Such groups are typically noncrystallizing. For example, the soft segments can be based upon noncrystallizing hydrocarbon backbones such as dimer acid derivatives, linked by urethane groups to short and/or medium chain length hydrocarbon moieties. The soft segments can also be derived from siloxane diols such as polydimethyl siloxane diol, polyether diols such as polytetramethylene ether glycols, polyester diols such as polyethylene/polypropylene adipate glycol polyester diol, and polycaprolactone polyester diol, and the like. Such diols can include methyl, phenyl, propyl, etc., substitution and can also include carbonol termination that may include any number of methylene units as desired. To improve the biocompatibility of a segmented polyurethane (SPU), 2-methacryloyloxyethyl phosphorylcholine (MPC) copolymer can be blended in the SPU by a solvent evaporation method from a homogeneous solution containing both SPU and MPC copolymer.

H. Solvents

It is understood that a solvent can be selected based upon its solubility properties vis-à-vis a selected pharmaceutically active agent, biologically active agent, polymer, or emulsion system.

In certain aspects, the organic solvent is a member selected from the group consisting of tetrahydrofuran, acetone, methylene chloride, chloroform, ether, hexane, pentane, petroleum ether, cresol, dichloroethane, ethyl acetate, methyl ethyl ketone, dioxane, propylene carbonate, and butyl acetate.

I. Supplementary Materials

In one aspect, the disclosed compositions and/or processes can further comprise a supplementary material. The supplementary material can be any supplementary material known to those of skill in the art and can be selected to modify the properties of the polymer fibers. For example, cellular adhesion can be improved by incorporation of soluble type I collagen into the disclosed compositions and/or processes by co-spinning the collagen from a solution of 1,1,1,3,3,3-hexafluoro-2-propanol using a dual needle system. The supplementary material can be added to the spinning solution to produce the polymer fibers. In a further aspect, the supplementary material can be added to the polymer fibers after spinning. In various aspects, the supplementary material comprises polymer fibers, a polymer network, or a coating.

In one aspect, the supplementary material comprises collagen, fibrin, chitin, laminin, polyethylene glycol, or a mixture thereof In a further aspect, the supplementary material comprises a synthetic peptide, a polysaccaride, a proteoglycan, or an extracellular matrix component, or a mixture thereof In various aspects, the supplementary material comprises polymer fibers. In further aspects, the supplementary material is nonpolymeric.

J. Additives

In one aspect, a composition or article can further comprise at least one additive. The additive can be any additive known to those of skill in the art and can be selected to modify the properties of the polymer fibers. The additive can be added to the spinning solution to produce the polymer fibers. In a further aspect, the additive can be added to the polymer fibers after spinning.

Various additives can be added to the emulsion, such as, for example, a surfactant, an emulsifier, and a stabilizer for impacting properties of emulsion such as stability, consistency, etc. Depending on ratios of first component to the second component, the emulsion can be a microemulsion.

In one aspect, the additive comprises a pharmaceutically active agent or a biologically active agent, for example, an antithrombogenic agent such as heparin. In this aspect, once the conduit is implanted into a subject, the additive can then be released from the porous conduit into a subject. In this aspect, the disclosed compositions can serve as a delivery system for one or more additives, for example pharmaceutically active agents.

K. Processes of Modulating Delivery Rates

The delivery rate—or release rate—of an additive, a pharmaceutically active agent, or a biologically active agent from polymer fibers can be modulated, or tailored, by the selection and co-electrospinning of two or more polymers. That is, two or more polymers can be co-electrospun into polymeric fibers and impregnated with an additive, a pharmaceutically active agent, or a biologically active agent, which is then released at a release rate when contacting a subject.

In one aspect, the invention relates to a process of modulating delivery rate of a pharmaceutically active agent or biologically active agent comprising the steps of providing a first amount of a first polymer comprising a first pharmaceutically active agent or biologically active agent, wherein the first pharmaceutically active agent or biologically active agent is capable of release from the first polymer at a first release rate when the first polymer is not co-electrospun; providing a second amount of a second polymer comprising a second pharmaceutically active agent or biologically active agent, wherein the second pharmaceutically active agent or biologically active agent is capable of release from the second polymer at a second release rate when the second polymer is not co-electrospun; and co-electrospinning the first polymer with the second polymer, thereby providing a co-electrospun polymeric fiber, wherein the first release rate is greater than the second release rate, wherein the first amount and the second amount are selected to provide a combined release rate for the co-electrospun polymeric fiber that is between the first release rate and the second release rate. In a further aspect, the first polymer and the second polymer can be the same or different. In a further aspect, the first pharmaceutically active agent or biologically active agent and the second pharmaceutically active agent or biologically active agent can be the same or different.

In a further aspect, the invention relates to a process of modulating delivery rate of a pharmaceutically active agent or biologically active agent comprising the steps of co-electrospinning a first amount of a first polymer with a second amount of a second polymer, thereby providing co-electrospun polymeric fibers, and impregnating the electrospun polymeric fibers with a pharmaceutically active agent or a biologically active agent, wherein the pharmaceutically active agent or the biologically active agent is capable of release from the first polymer at a first release rate when the first polymer is not co-electrospun and capable of release from the second polymer at a second release rate when the second polymer is not co-electrospun; wherein the first release rate is greater than the second release rate; wherein the first amount and the second amount are selected to provide a combined release rate for the co-electrospun polymeric fiber that is between the first release rate and the second release rate.

L. Processes for Delivering Pharmaceutically/Biologically Active Agents

In one aspect, the invention relates to a process of delivering a pharmaceutically active agent or biologically active agent, the method comprising the steps of providing a co-electrospun polymeric fiber comprising a first polymer comprising a first pharmaceutically active agent or biologically active agent, wherein the first pharmaceutically active agent or biologically active agent is capable of release from the first polymer at a first release rate when the first polymer is not co-electrospun; and a second polymer comprising a second pharmaceutically active agent or biologically active agent, wherein the second pharmaceutically active agent or biologically active agent is capable of release from the second polymer at a second release rate when the second polymer is not co-electrospun, wherein the first release rate is greater than the second release rate, wherein the first pharmaceutically active agent or biologically active agent and the second pharmaceutically active agent or biologically active agent are released from the co-electrospun polymeric fibers at a combined release rate between the first release rate and the second release rate; and contacting the co-electrospun polymeric fiber with a subject, thereby delivering the first pharmaceutically active agent or biologically active agent and the second pharmaceutically active agent or biologically active agent at a combined release rate. The providing step can comprise, for example, co-spinning the first polymer and the second polymer. In one aspect, the contacting step can be implantation or topical administration.

In a further aspect, the process can further comprise the step of removing the co-electrospun polymeric fiber from the subject. In one aspect, the subject is a mammal, for example a human, for example a patient.

M. Modulation of Drug Delivery Characteristics to Electrospun Fibers

The disclosed compositions and processes demonstrate that key properties of electrospun fibers, such as diameter, can be modulated by electrospinning multiphasic systems. In addition to changing morphological aspects of the fibers, the disclosed compositions and processes allow for the introduction of bioactive moieties (e.g., pharmaceutically active agents and/or biologically active agents) in a safe and simple manner through incorporation into the aqueous phase. Imparting drug delivery characteristics to electrospun fibrous systems can expand its repertoire of applications in drug delivery, scaffold design, and regenerative medicine. Factors that affect the release of doxycycline, an antibiotic; fluorescein isothiocyanate conjugated albumin (FITC-BSA), a model protein; and horseradish peroxidase (HRP), a model enzyme, from poly(L-lactic acid) (PLA) and poly(ether-urethane) (PU) fibers have been analyzed herein. PLA and PU were selected as model polymer systems due in part to their widespread use in current medical products and contrasting physicochemical properties.

In certain examples, doxycycline release was monitored for 10 days with detectable release during the first 100 hours of the experiments. Over 90% of the release occurred within 6 hours of hydration. Cyclodextrin-complexed doxycycline released at half the rate as uncomplexed antibiotic. FITC-BSA release was monitored with steady release occurring for over 350 hours. HRP followed a similar release profile, with enzyme activity being most prevalent during the first 6 hours of the release study with measurable activity up to 24 hours after hydrating the electrospun fibers. Enzymatic activity was quantified by tetramethybenzidine conversion and indicates that proteins can be released in their native, non-denatured form from electrospun meshes.

Electrospun meshes have great potential for biomedical applications as tissue engineering scaffolds and drug delivery devices. A wide variety of polymers—ranging from natural (1) and recombinant proteins (2), polysaccharides (3), and degradable and non-degradable synthetic polymers—have been electrospun by various research groups. Researchers have also explored methods to modulate fiber morphology and mesh composition with great flexibility and reproducibility. The combination of these provides great control over the physicochemical properties of electrospun meshes. It has been shown that emulsions can provide mechanism to control fiber diameter. Designing approaches to release bioactive agents in a controlled fashion is not only powerful in a drug delivery aspect, but also in a tissue engineering regard. In their native environment, cells are presented with information in the form of physical cues as well as chemical signals. The rich mixture of soluble and insoluble molecules can affect how a cell behaves. ES provides the ability to create fibers on the same length scale as natural ECM components (from tens of nanometers to the micron-size range) using a wide variety of biologically relevant materials. While drug release from nanofibers has been previously demonstrated, the disclosed emulsion techniques provide methods to release drugs from an aqueous environment, which can be necessary for the delivery of sensitive proteins or peptides, using a simple ES technique that does not require any modifications to standard ES equipment.

The disclosed compositions and processes demonstrate the successful encapsulation of hydrophilic compounds in electrospun polymer fibers and the subsequent release upon hydration of the electrospun mesh. Other labs have demonstrated that it is possible to release drugs from single phase fibers, through incorporation of hydrophobic molecules or by solubilizing the compound with a miscible solvent. However, such approaches can be detrimental to certain therapeutics such as proteins, which can denature in organic solvent. Researchers have also been able to electrospin multiphase fibers, though this required special modifications to their electrospinning apparatus.

The disclosed emulsion technique is unique in the sense that it provides an aqueous platform to deliver hydrophilic drugs and proteins while adding no additional complexity to the electrospinning equipment. The disclosed compositions and processes electrospun meshes are viable candidates for creating well defined tissue engineering microenvironments or drug delivery devices, in part because reproducible controlled release of compounds are demonstrated. The disclosed compositions and processes provide multiphasic electrospinning systems to fabricate drug carrying nanofibers. It is shown that active enzyme can be successfully survive the electrospinning process using the emulsion technique. It has also been shown that release profiles can be modified by loading additional components into the aqueous phase of the emulsion to accelerate or retard diffusion of the drug. Cyclodextrin-complexed doxycycline, for example, was released at a slower rate than free doxycycline, indicating that supramolecular complexes between drugs and other molecules can modulate release. This is further supported by disclosed data showing the relatively large FITC-BSA complex had much slower release rates than the smaller doxycycline molecules.

In the disclosed compositions and processes, bioactive molecules (doxycycline) and model compounds (HRP, FITC-BSA) have been encapsulated in synthetic polymer fiber and release has been quantified. The disclosed data show that small molecules such as doxycycline are released rapidly (>6 hours) from the hydrated mesh while larger molecules FITC-BSA show release past 350 hours. The disclosed data also demonstrates that release rates can be modified. That is, co-spinning multiple fibers has an additive effect on release kinetics. Also, adding excipient in the aqueous phase can retard release kinetics of small molecules.

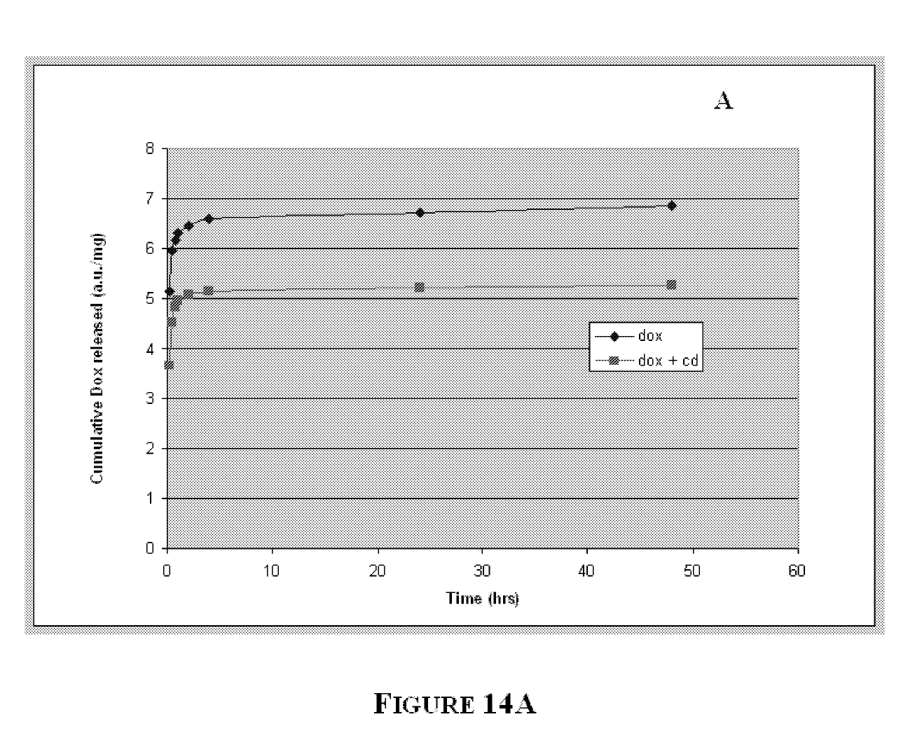

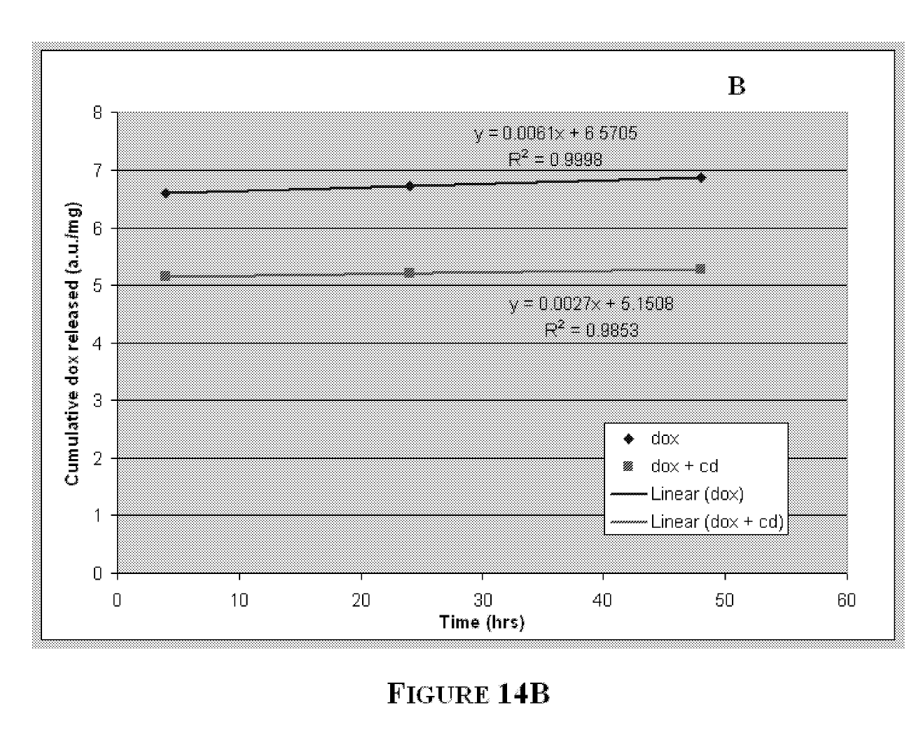

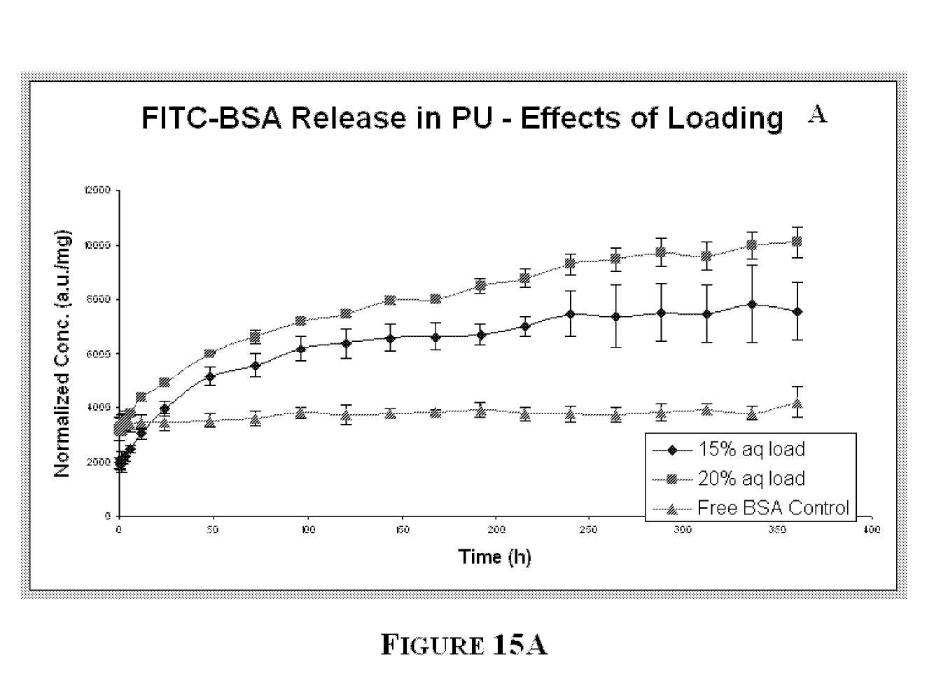

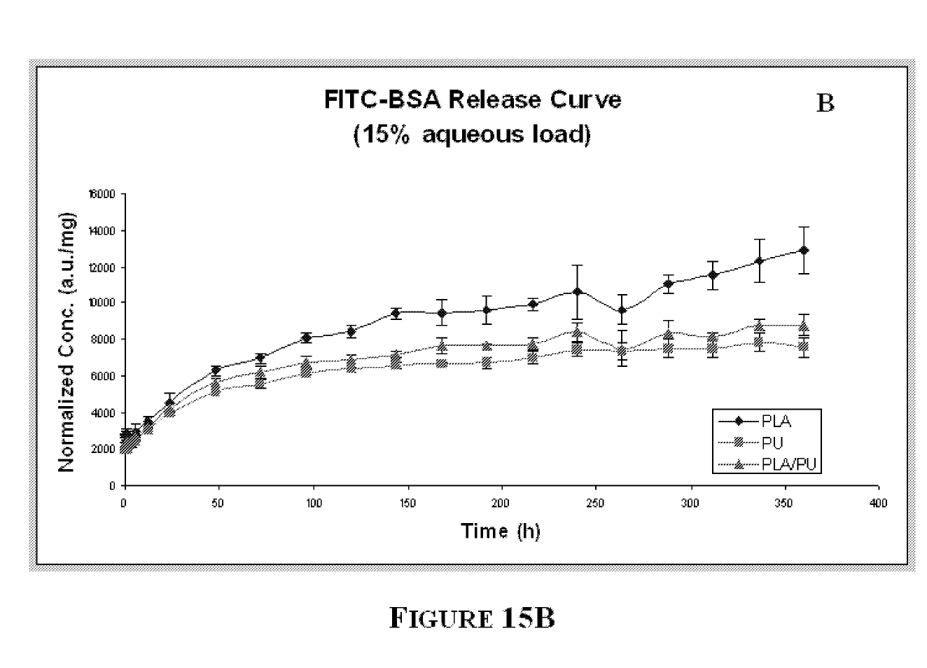

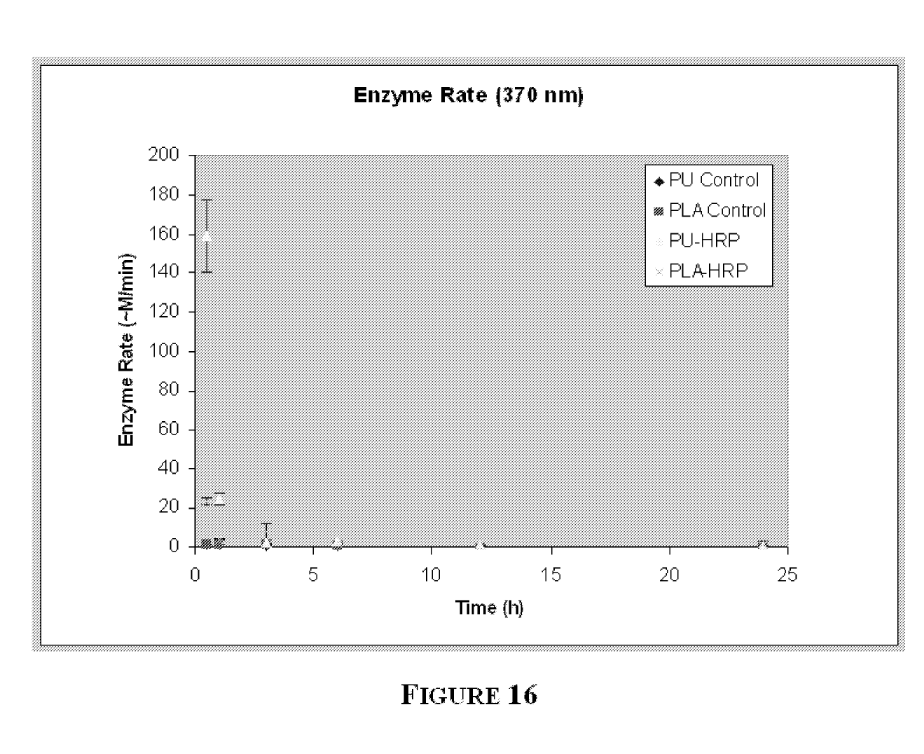

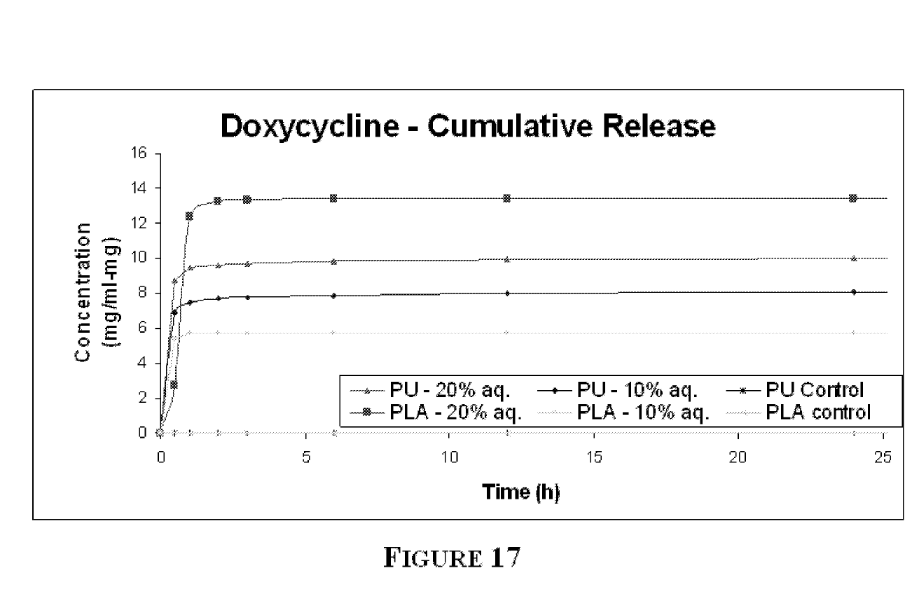

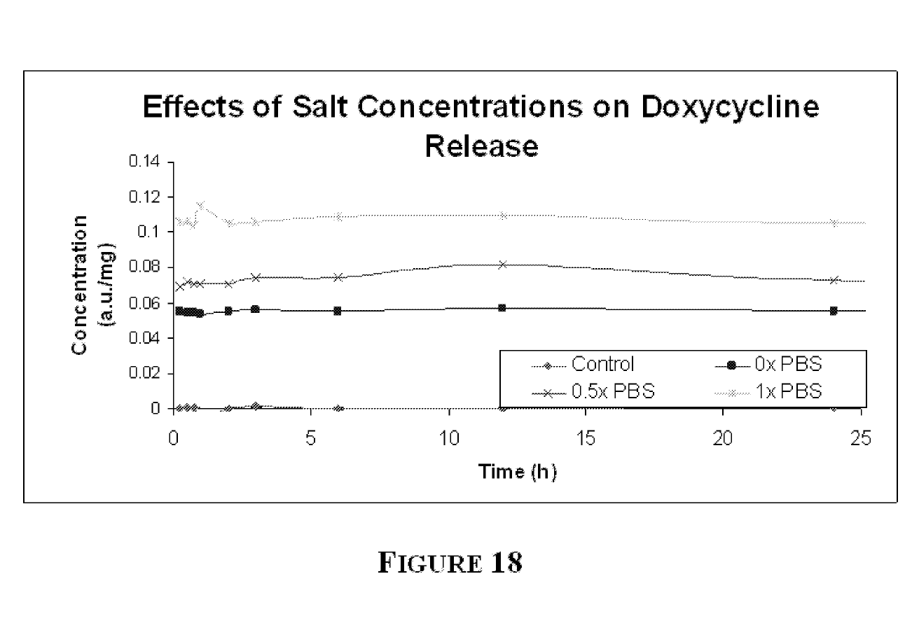

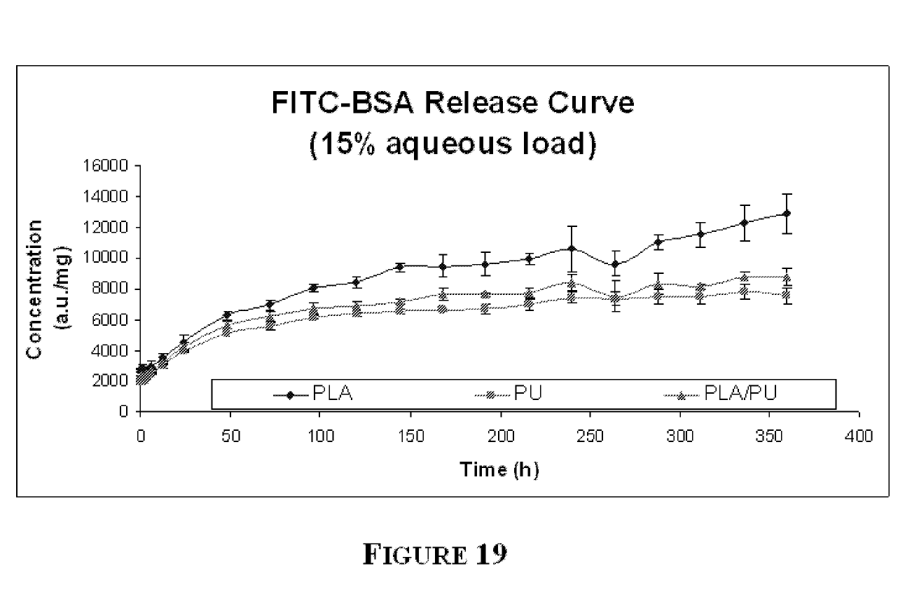

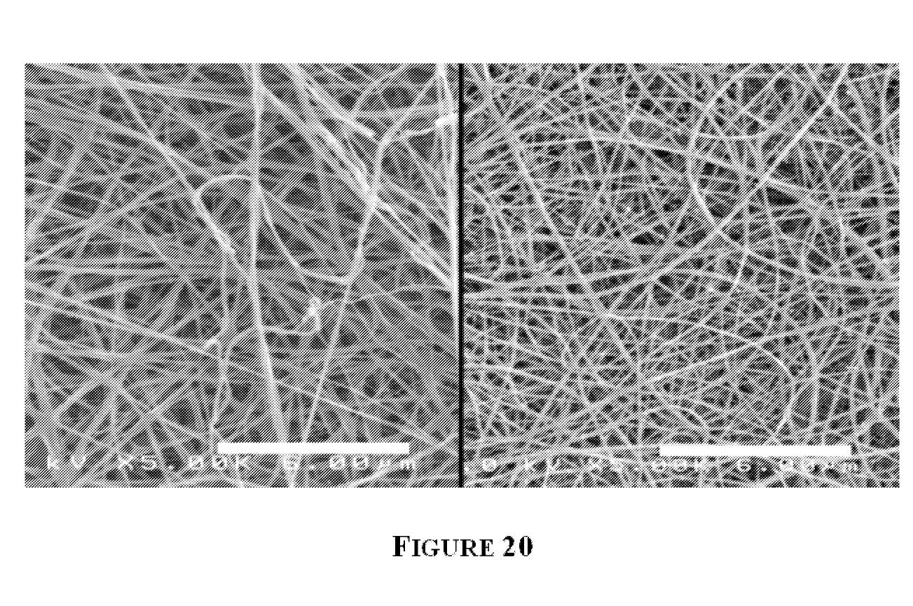

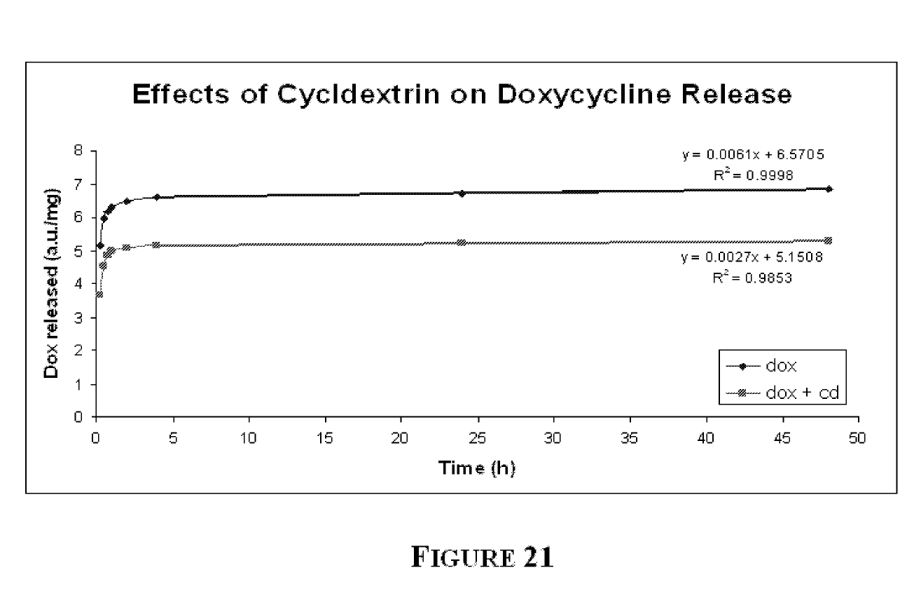

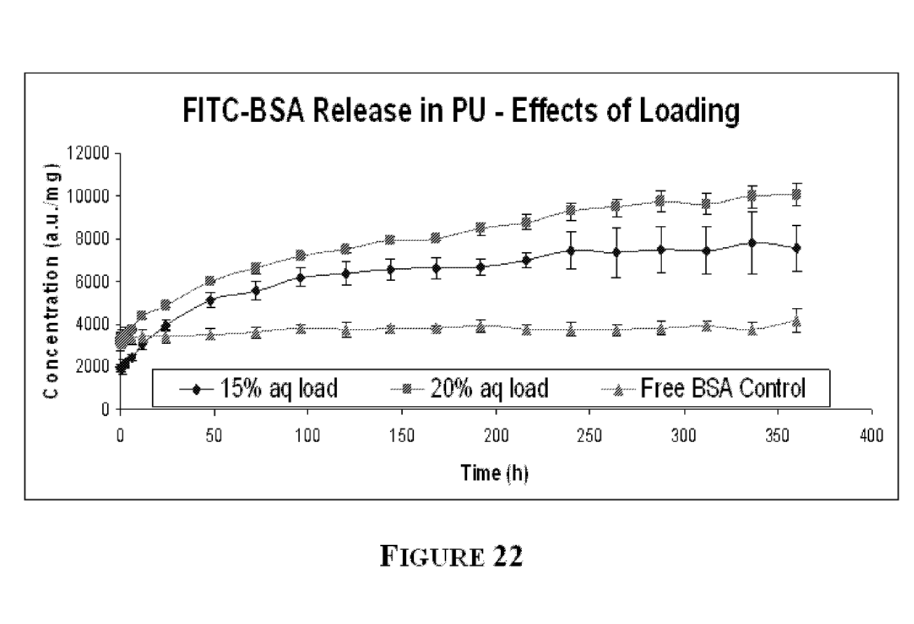

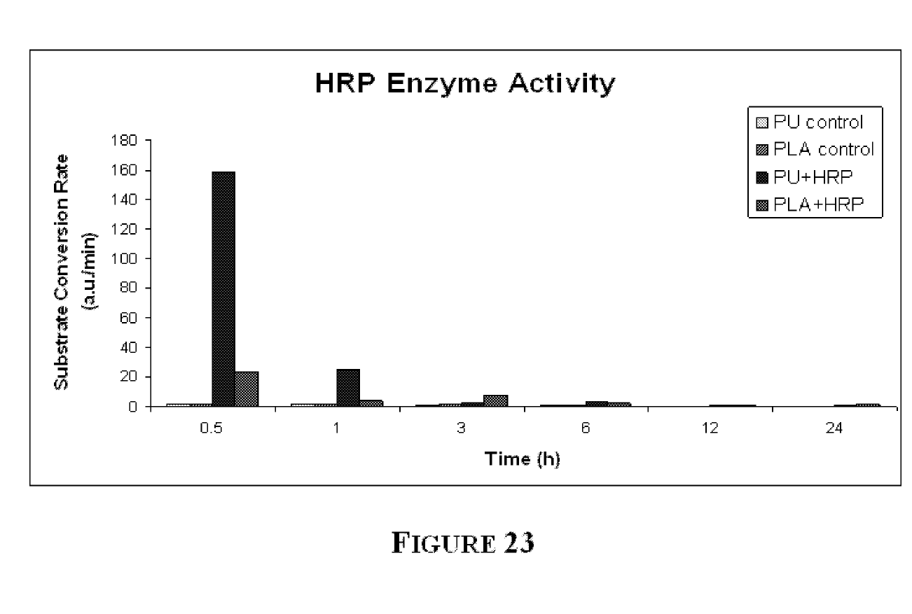

FIG. 14-FIG. 16 show the release of various molecules ranging from a small molecule antibiotic Doxycyline, proteins Horse Radish Peroxidase (HRP, MW ˜30 KDa) and Bovine Serum Albumin (BSA, MW ˜65 KDa) for polyurethane (PU) and poly(L-lactic acid) (PLA) from emulsion-based electrospun fibers.

N. Experimental

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Materials

Poly(L-lactic acid) (MW=300,000) (PLA) was purchased from Polysciences, Inc. (Warrington, Pa.). Polyvinyl alcohol) (MW=IO5OOO, 85% hydrolyzed) (PVA) and 1-methyl-2-pyrrolidinone (99.5%) (NMP) were purchased from Aldrich Chemical Co (Milwaukee, Wis.). Chloroform (HPLC grade, 99.8%) was purchased from Fisher Scientific (Pittsburgh, Pa.). Poly(acrylic acid) coated Silica colloids 500 nm in diameter were produced by a sol-gel process. All chemicals were used as received without further purification unless otherwise noted.

2. Preparation of Polymer Solutions

Water-in-oil (W/O) emulsion of PLA was prepared by emulsifying a 2% stock of PLA in chloroform with 5% PVA solution in water and a fixed volume of NMP. NMP was added to the mixture to serve as a phase compatibilizer (NMP is soluble in both water and chloroform) and to retard the evaporation of chloroform (oil phase). To aid in the analysis of the evolution of fiber morphology and get an insight into the mechanism of fiber formation, silica colloids (<1% v/v) were added to some of the formulations. Components were metered using an Eppendorf pipette, mixed by vortexing and sonicated for 45 seconds (20 KHz, Vibra Cell, Sonic Systems) to ensure full emulsification.

3. Electrospinning

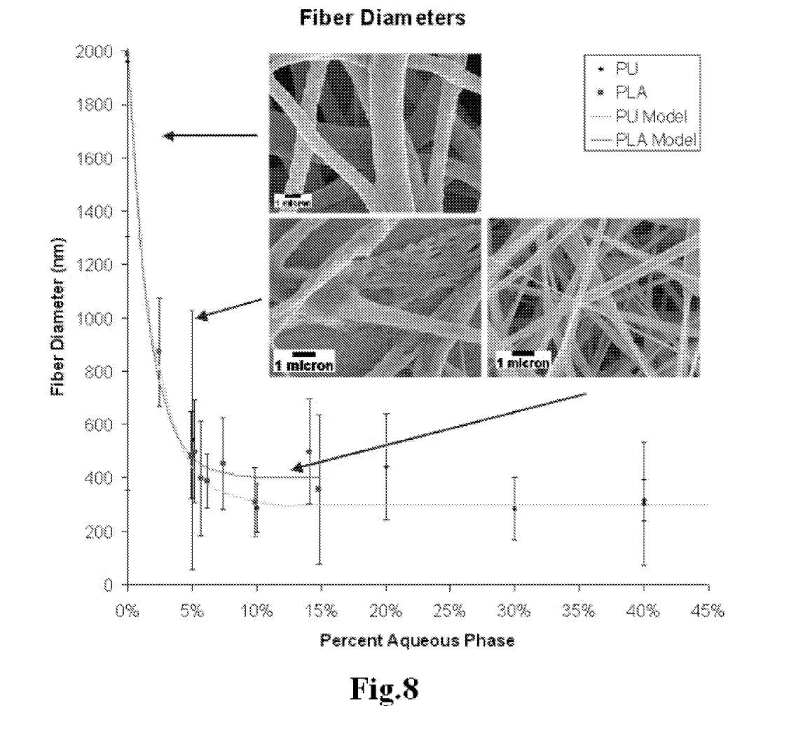

A series of solutions using 15% (w/w) PU solution with varying amounts of aqueous phase was electrospun (17 kV applied voltage, 20 cm tip-to-target distance, 0.1 ml/min, 16 gauge needle) to produce the fiber diameter versus percent aqueous phase curve shown in FIG. 8. The emulsion mechanism has the most drastic effects in the low aqueous concentration ranges (0-5%), consistent with rheological data that suggests that the most dramatic effect in shear thinning comes with small additions of the second immiscible phase. There is an order of magnitude decrease in average fiber diameter (from 1960 nm to 540 nm) with the addition of 5% (w/w) of poly(vinyl alcohol) (PVA) solution. Without wishing to be bound by theory, it is believed that one possible mechanism for this large decrease in fiber diameter is the fact that the emulsion exhibited enhanced shear thinning at the tip of the “Taylor cone,” allowing for a greater reduction in the cone diameter. Towards the end of the “Taylor cone” the less volatile aqueous phase occupies a greater volume fraction of the jet, allowing for greater thinning/deformation of the cone compared to the highly viscous polymer/organic solvent gel.

A similar experiment was performed using a poly(1-lactic acid) (PLA) system. Using a base solution of 2% (w/w) PLA dissolved in chloroform, varying amounts of PVA/water and N-methylpyrrolidone (NMP) were emulsified with the polymer solution and electrospun (25 kV applied voltage, 15 cm tip-to-target distance, gravity fed spinneret, 16 gauge needle). Diameter versus percent aqueous phase is shown in FIG. 8. The PLA system showed a similar order of magnitude decrease (from 2000 nm to 490 nm) with the addition of just 5% aqueous phase.

For both systems, the decrease of fiber diameter with increasing aqueous content leveled out to 300 nm and 400 nm for PU and PLA, respectively. There was an upper limit to the amount of aqueous phase that could be added to the solutions since high aqueous containing solutions tended to electrospray. However, this asymptotic behavior of the fiber diameter indicates that the mechanism is largely a shear thinning mechanism, rather than a concentration effect. While apparent viscosities of the solutions increased with aqueous concentration, all emulsions showed a limiting shear thinning viscosity value.

PLA and polyurethane (PU) emulsions were tested using a Brookfield Viscometer (Model LVDV-II+, Middleboro, Mass.) with a cone and plate spindle (model CPE-40, 0.8° cone spindle, 0.5 ml sample volume) at room temperature. Rheological data obtained confirmed two principles of our proposed mechanism. An increase in apparent viscosity allowed for the electrospinning of low polymer concentrations while more pronounced shear thinning at high shear rates allowed for the formation of thinner fibers.

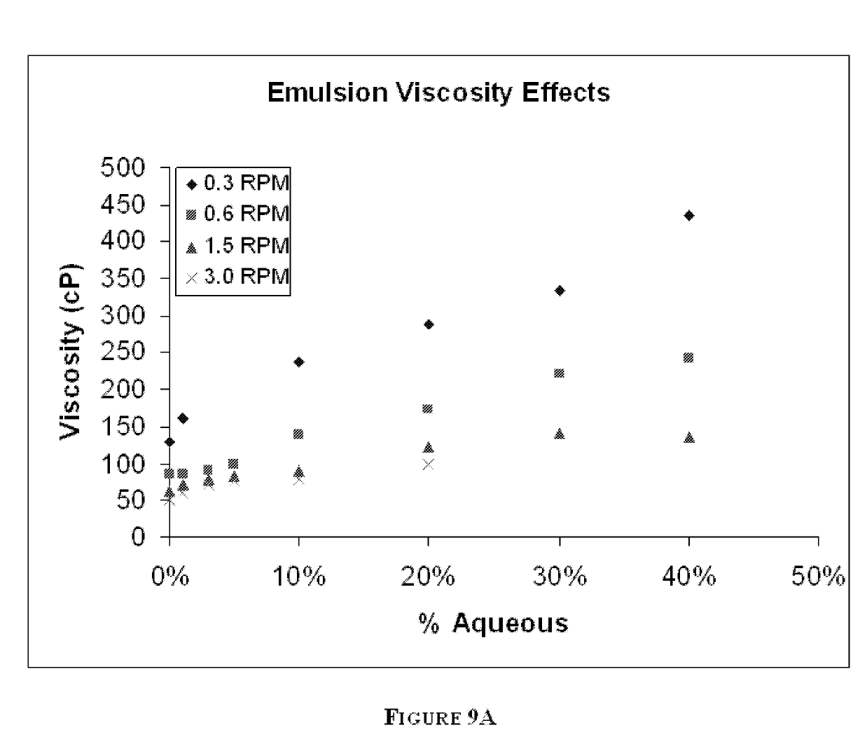

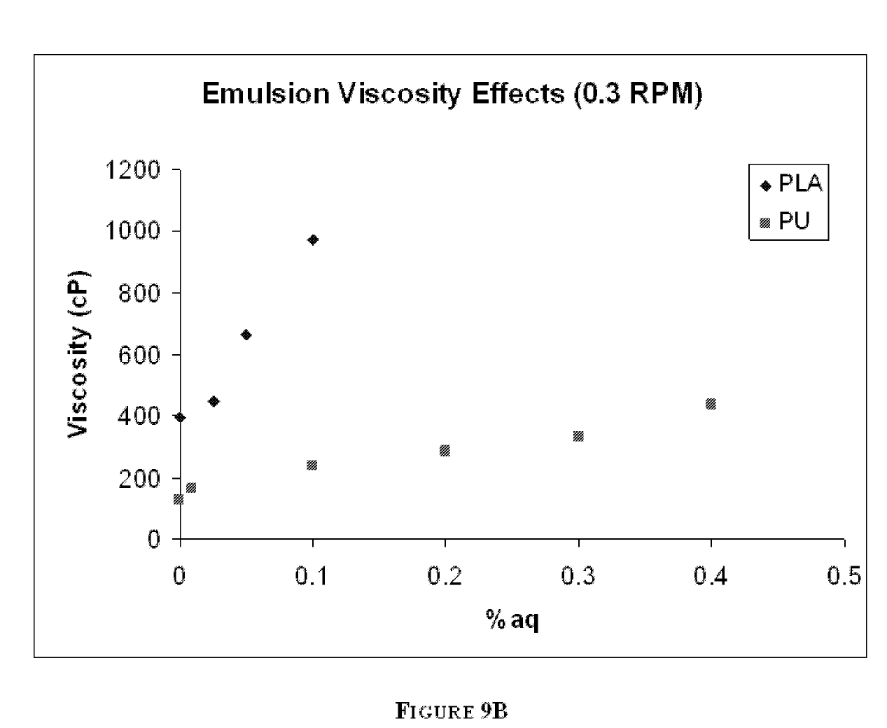

Samples of 6% PU (w/w) dissolved in THF/chloroform (equal volumetric ratios of each solvent) were emulsified with varying amounts of 10% PVA/water (w/v). FIG. 9 shows how increasing aqueous content of the solution increased the viscosity of the electrospinning solution. A 10% aqueous emulsion had over a two-fold increase in apparent viscosity of the solution (128.7 cP to 435.2 cP) at the slowest shear rate (0.3 RPM). This dramatic increase in apparent viscosity explains how the present methods electrospin, rather than electrospray, dilute polymer solutions. Similar results were obtained with the PLA system.

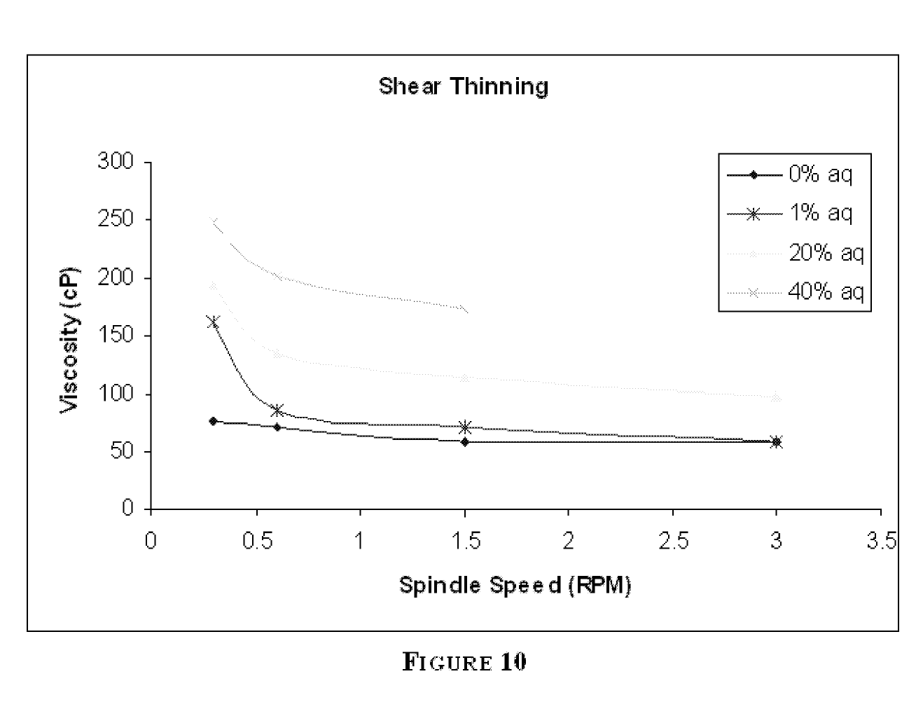

Shear thinning was tested using four different spindle rotational speeds (0.3, 0.6, 1.5, 3.0 RPM) at a number of different aqueous contents. For all aqueous concentrations, shear thinning was most pronounced at slower shear rates (i.e., the transition between 0.3 RPM and 0.6 RPM). As spindle rotational speed increased, the viscosity of the solution approached a limiting value (FIG. 10).

In addition to changing the rheological properties of the electrospinning solution, a secondary effect of adding multiple phases to the solution was that the less volatile liquid phase acted as a template during fiber solidification and formation. This effect produced fibers with varying morphologies ranging from round to porous to ribbon-like.

This effect was most evident in the PLA system. PLA fibers spun with low aqueous concentrations (<5% by volume) were predominately round in morphology. However, as the amount of aqueous phase increased in this range, fiber porosity increased. Without wishing to be bound by theory, it is believed that these pores were likely formed as the PLA solidified around the aqueous droplets during the electrospinning process. As the aqueous phase evaporated after the fibers were formed, they left behind open pores in the polymer matrix.

As aqueous concentration was increased above 5%, the appearance of ribbon-like fibers become more predominant. The likely mechanism for the formation of these fibers is due to the collapse of hollow PLA tubes. As the aqueous phase is increased, there is a greater volume fraction of water during the fiber formation process, which leads to a greater templating effect of the aqueous phase. This theory was tested by dispersing colloidal silica in the aqueous phase. Due to silica's hydrophilicity, the silica particles could be used to track the migration of the aqueous phase. The silica particles were sequestered in the polymer matrix and took on a pearl-chain configuration as particles were lined up in close proximity to one another.

While ribbon formation did not dominate at high aqueous concentrations for the PU and PEVA electrospun fibers, it is believed that the elastic properties of the polymer contribute to the amount the aqueous phase can act as a template during fiber formation. Both PU and PEVA have strong elastomeric properties. This recoverable elastic deformation of the polymer could have prevented the collapse of the hollow tubes. PLA has a much higher elastic modulus and less recoverable deformation. In fact, many of the PLA ribbons appear like they were split polymer tubes.

In addition, solvent compatibility also plays a role in this process. The particular PU used in this example was only dissolvable in THF, which is fully miscible with water. As a result, an additional organic solvent, chloroform in this case, was used to create an emulsion. However, the partitioning and phase separation of the aqueous phase was not fully studied and may have contributed to the round fibers seen with most of the PU samples.

For polymers that did not exhibit flat fiber morphology at high aqueous concentrations (PU, PEVA, and PVA not shown), larger droplets of water were encapsulated. The presence of the water phase was confirmed by labeling the water phase with fluoroscein. These features were visible on an optical microscope, indicating that the water droplet size was much larger than the actual polymer fiber diameter in that particular PEVA system.

4. Co-Spinning

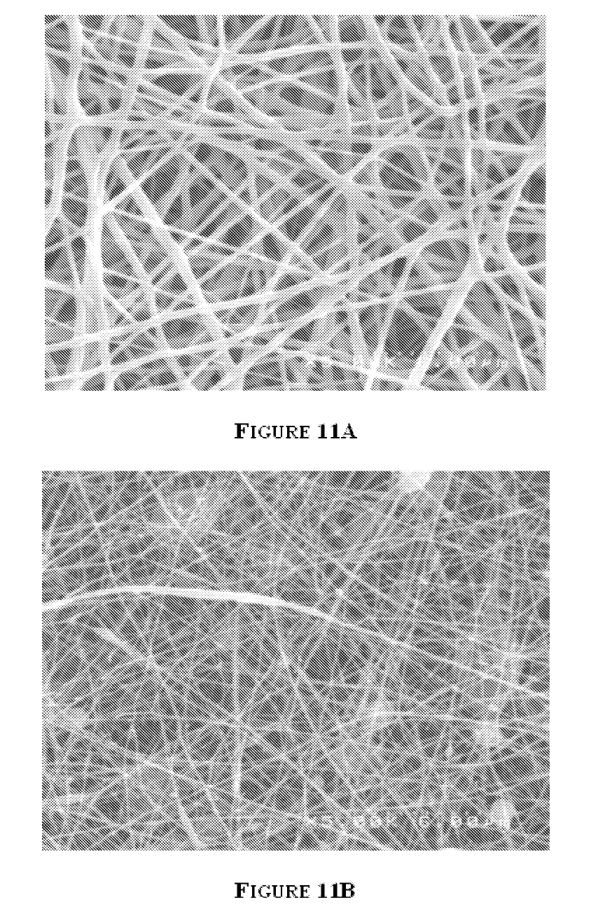

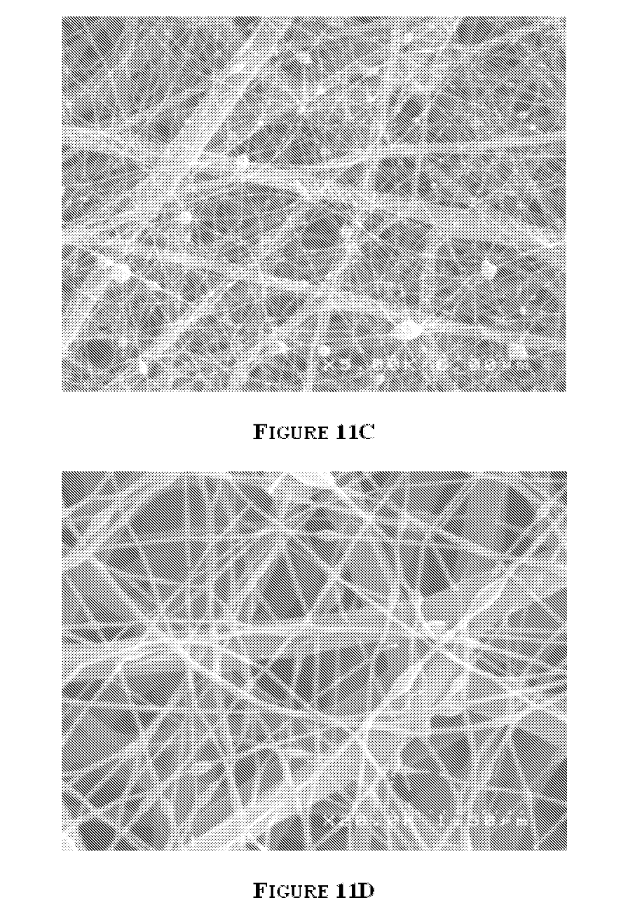

Co-spinning compositions with both a degradable/biologically remodelable and non-degradable polymer using a two-needle cospinning approach was investigated. Compositions containing both PU and bovine type I collagen (electrospun out of a 40 mg/ml solution in 1,1,1,3,3,3-hexafluoro-2-propanol) have been produced using the methods of the invention (FIG. 11). By cospinning PU and collagen into the composition, it is possible to utilize strengths of both materials. PU provides a strong, elastic framework for the composition, lending it immediate mechanical integrity as well as compliance. Collagen provides a natural extracellular matrix (ECM) that can aid in cellular attachment, proliferation, and differentiation, but typically lacks adequate mechanical properties to be useful or functional. The addition of additional ECM proteins or other synthetic polymers allow further tuning of the physical and chemical properties of the conduit.

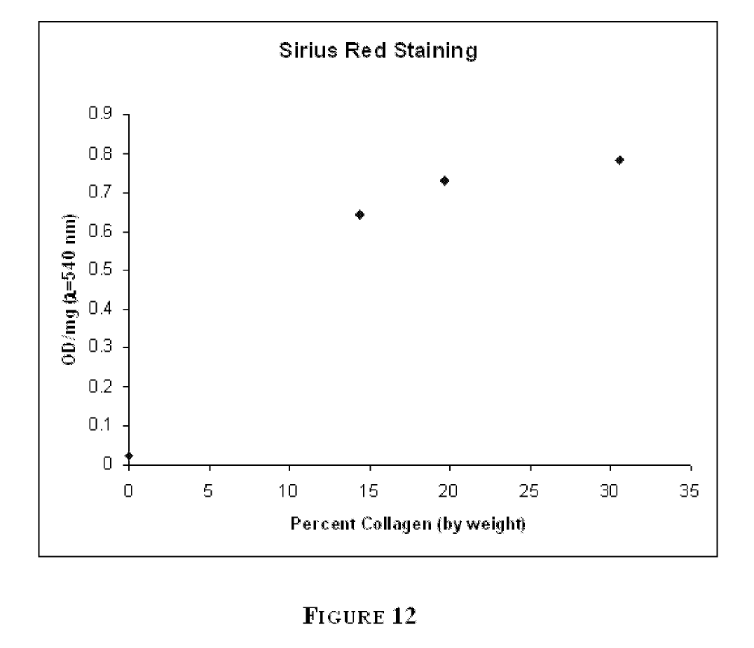

The presence of cospun collagen was confirmed both by SEM (as seen in FIG. 11) and spectrophotometrically. Samples containing different weight percentages of collagen were prepared by varying the flow rate of each polymer solution. Samples ranging from 0% collagen to 30% collagen were prepared. Compositions were fixed with gluteraldehyde to stabilize collagen fibers. Samples were subsequently stained with Sirius red/picric acid solution and rinsed to remove excess dye. Dye bound to the collagen fibers was solubilized in NaOH solution overnight and the resulting supernatant was analyzed spectrophotometrically. Optical density/absorbance was measured at 540 nm and normalized to sample mass. As expected, normalized optical density of the solubilized dye increased with collagen content (FIG. 12).

While not wishing to be bound by theory, it is believed that the increased interaction between multiple phases can create a higher viscosity than the component parts individually. This phenomenon can be particularly evident in the polyurethane/chloroform:THF (1:1) system of the present methods (see Table 1). Conventional methods require a polymer concentration of at least 12% (w/w) to produce fibers—lower polymer concentrations (5%, 10%, 11%) electrospray under the same conditions. In contrast, with the emulsion technique of the invention, it was possible to spin a 7.5% (w/w) PU solution with as little as 5% aqueous phase emulsified into the solution.

TABLE 1

| | | | | | | Polymer |
|---|---|---|---|---|---|---|
| | | | | Tip-to- | | concentration |
| | Organic | Phase | Applied | target | Molecular | (in organic |
| Polymer | Solvent | compatibilizer | Voltage | distance | Weight | solvent) |
| poly(l-lactic | CHCl₃ | NMP | 25 kV | 15 cm | 300 kDa | 2% |
| acid) | | | | | | |
| polyurethane | CHCl₃/THF | none | 17 kV | 20 cm | 130 kDa | 6% |
| poly(ethylene | CH₂Cl₂ | NMP | 25 kV | 15 cm | 70 kDa | 7.5% |
| co vinyl | | | | | | |
| acetate) | | | | | | |

5. Electrospinning of PLA Fibers

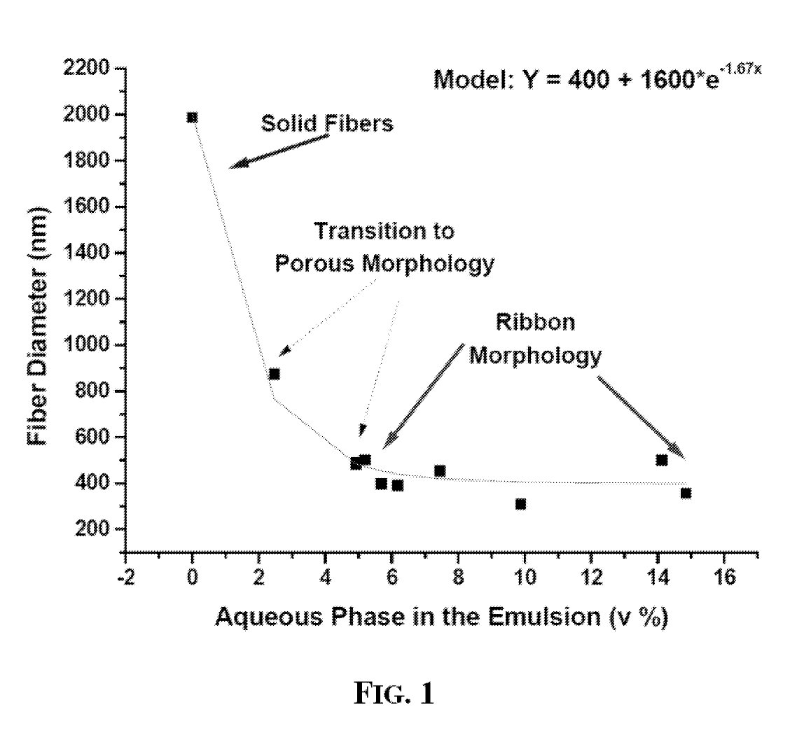

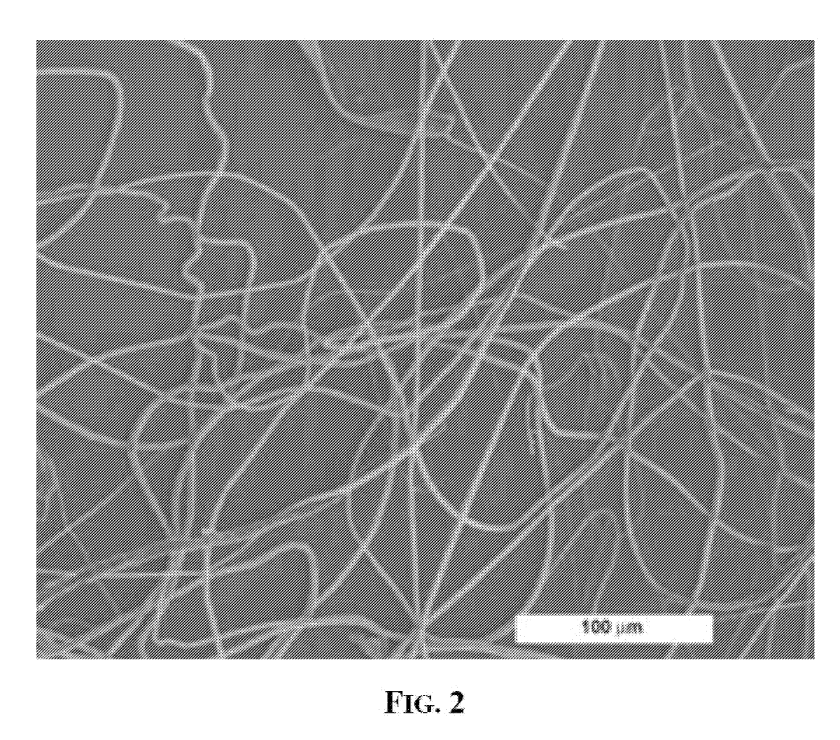

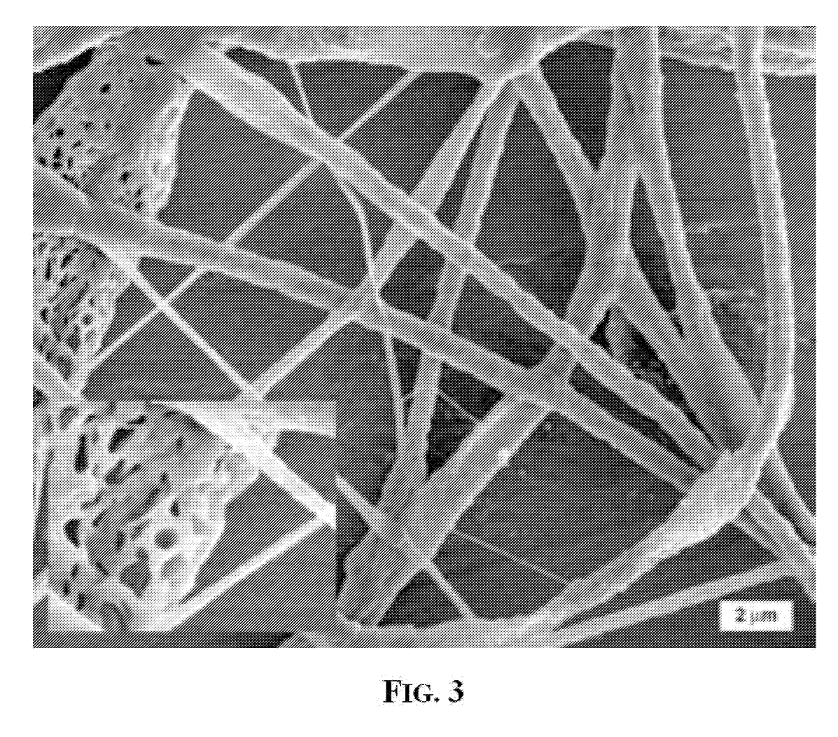

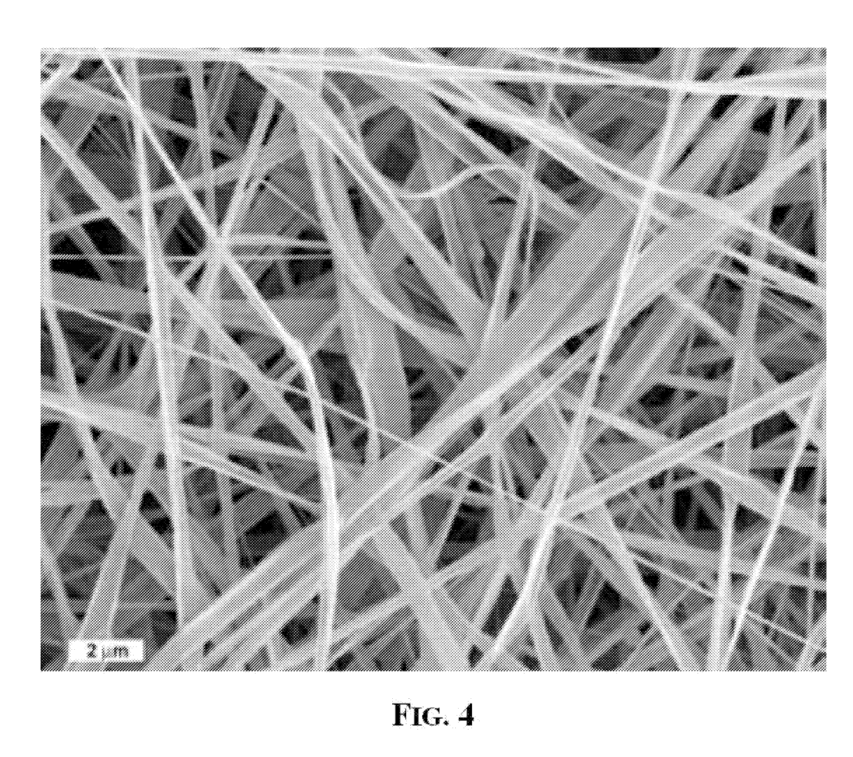

The polymer solution (typical volume 1 ml) was loaded into a 3 ml syringe fitted with a 16-gauge blunt tip needle. The syringe was mounted on a ring stand at a 45° angle below horizontal. The needle was connected to a high voltage power supply (Gamma High Voltage Research, Ormond Beach, Fla.). The counter electrode was connected to an aluminum foil (collecting target) placed at a distance of 15 cm away from the tip of the needle. The bias between each plate was then slowly increased until the eruption of the “Taylor Cone” and was then set at 25 kV. Fibers were collected on the aluminum foil until the solution was fully dispensed. Electrospun fibers were imaged using a JEOL 6300FV field emission scanning electron microscope at an acceleration voltage of 10 KeV. Samples were mounted onto aluminum stubs using conductive carbon tape and then sputter coated with Pd—C to minimize charging. TIFF files of the images were then imported into Scion Image (NlH, Bethesda, Md.) for analysis. W/O emulsions of PLA dissolved in a chloroform/NMP mixture and water, stabilized by PVA, were used as a model two-phase system to study its effect on fiber morphology in the ES process. The choice of this system was driven by two considerations, namely, easy adaptability to biomedical applications and biocompatibility of the non-volatile components. Solutions containing up to 15% aqueous phase were successfully electrospun without any disruption of the fiber morphology. However, solutions that contained greater than 20% by volume of aqueous phase tended to spray as droplets suggesting the onset of instability of the “Taylor Cone.” It was observed that by varying the volume fraction of the aqueous phase, the morphology and diameter of PLA fibers could be significantly impacted. In general, increasing the volume fraction of the aqueous phase yielded fibers with smaller diameters (FIG. 1). Without wishing to be bound by theory, one contributing factor can be the lower volume fraction of polymer at higher aqueous phase concentrations. Rheological effects are most likely the dominant component of the fiber-thinning process. However, no correlation was observed between PVA concentration and fiber diameter. A synergetic effect was observed, wherein an order of magnitude change in fiber diameter can be achieved with the introduction of a small volume fraction of aqueous phase. The fiber diameter data can be fitted to an exponential decay process, which is consistent with a trend one may observe with respect to the stability of emulsions. The typical fiber morphology obtained in the ES process is that of a circular rod (FIG. 2). However, in this invention fiber morphology can be varied from round spaghetti-like, to porous (FIG. 3), to flat ribbon-like fibers (FIG. 4) without varying the conditions of the ES process, namely the bias lent by selecting appropriate emulsion compositions. SEM analyses of the fibers reveal that the transition from round to porous fibers occurs in the range of 2-5% volume fraction of aqueous phase in the emulsion. Above 5% volume fraction of aqueous phase, fibers with a flat-ribbon morphology are obtained. This transition may be explained as follows. At lower aqueous phase volume fractions, the emulsion droplets are relatively stable and there is no further segregation for the entire duration of the ES process. As the emulsion solution is propelled towards the target the polymer fraction, which constitutes the vast majority undergoes solidification due to the evaporation of the volatile organic phase (chloroform) and the resulting fiber stretches as it approaches the target, while the aqueous phase remains entrapped within the rapidly solidifying polymer (oil) phase. The aqueous droplets become regions of instability toward the later stage of solidification as it constitutes a larger portion of the liquid phase, and a surface tension driven phase segregation process can result yielding porous fibers upon the evaporation of the aqueous component. At still higher volume fractions of aqueous phase, the stability of the emulsion is rather poor even at the early stage of ES and solidification and this leads to rapid phase segregation and the encapsulation of larger water droplets within the solidifying polymer phase. As the polymer skin evolves, the aqueous phase coalesce to yield a structure similar to a water filled balloon or a garden hose. The polymer skin eventually collapses, probably after partial evaporation of the entrapped aqueous phase, because of buckling instability in bending a thin wall tube. This yields fibers with flat, ribbon-like morphologies. This mechanism has also been verified through indirect observations in systems containing silica colloids.

6. Collection of Fibers

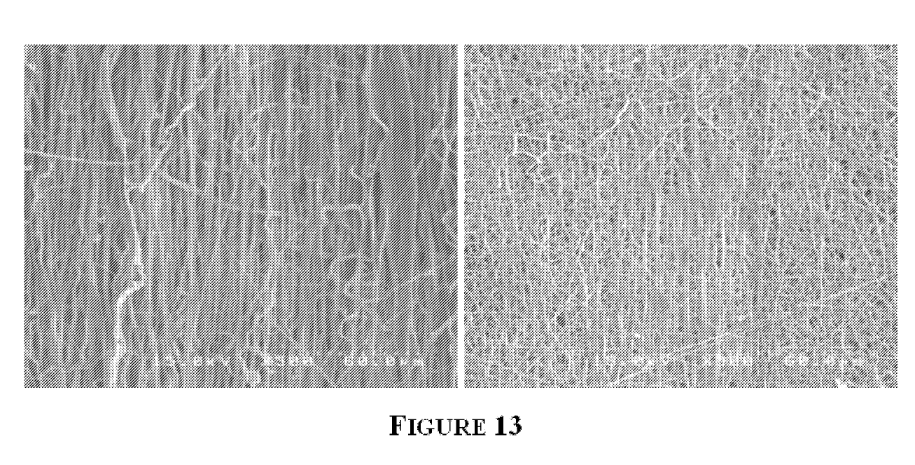

Researchers have been able to align electrospun fibers over short distances (<5 mm) using rotating collecting targets with narrow collecting surfaces and high rotational rates (X. M. Mo et al.; A. Theron, et al.) or by using a multiple electrode configuration (D. Li et al.). Polymeric fibers can be collected on a collection surface, for example, on a mandrel rotating at 7500 rpm and being translated laterally by an additional electric motor. The surface can be grounded opposite a charged needle/polymer solution at 17 kV. Alignment of fibers can be achieved by using short (˜5 cm) electrospinning tip-to-target distances. Without wishing to be bound by theory, it is believed that this shorter distance reduces the time of flight of the polymer fiber, thereby reducing the whipping motion of the Taylor cone. Substantially aligned polyurethane fibers were collected (FIG. 13).

Alignment of polymer nanofiber was confirmed using a Hitachi S-4200 Scanning Electron Microscope (SEM). Samples prepared were composed of a polyurethane (PU)/poly(vinyl alcohol) (PVA) solution using an emulsion system. The emulsion system allowed tailoring of fiber diameter as well as electrospinning viscosity.

7. Drug Delivery

a. Electrospinning Conditions

Polymers were dissolved in organic solvent and emulsified with a H₂O/Poly(vinyl alcohol) mixture with the molecule to be released dispersed in the aqueous phase. Polyurethane (PU) was dissolved at 6 wt % in a mixture of chloroform and tetrahydrofuran. PLA was dissolved at 2 wt % in chloroform. Drugs were loaded into the PVA/Water mixture with aqueous volume fractions ranging from 10-20 vol %. Emulsions were loaded into plastic syringe fitted with a blunt tip 18-gauge needle and placed into a syringe pump.

b. Release Parameters

Circular discs were punched out from the aluminum foil and polymer fibers removed from Al-backing. Samples placed in centrifuge tubes individual glass vials with PBS. Samples degraded in at room temperature (20° C.) on a rotisserie. The buffer was sampled at given intervals. Depending on release molecule, different spectrophotometric techniques were used to measure release profiles.

c. Electrospinning

Polymers are produced by applying a strong electric field (1-3 kV/cm) between a polymer solution and target. The voltage draws a cone from the spinneret until the onset of instability. As the fiber rapidly whips and thins, solvent evaporates, leaving an nanoscale polymer fiber.

d. Polymer Properties

Poly(1-lactic acid): MW 300 kDa. Poly(ether urethane): MW 80 kDa. Poly(vinyl alcohol): MW 10 kDa, 90% hydrolyzed.

e. Drug Properties

Doxycycline Hyclate: MW 513 Da. FITC-Bovine Serum Albumin: 66 kDa. Cyclodextrin: 1 kDa. Horseradish Peroxidase: 40 kDa.

f. Doxycycline & Doxycycline-Cyclodextrin Mixture

The concentration was determined spectrophotometrically at λ=267 nm and 351 nm. Fiber meshes were placed in 400 μl of PBS, which was replaced every sample point. Average concentration plotted over 48 hour time course (n=3).

g. Horseradish Peroxidase

Enzyme activity assayed by 3,3,5,5-tetramethylbenzidine (TMB) assay. Samples were placed in 10 ml of PBS and 200 μl of buffer was used to run the assay and not replaced (total amount withdrawn from tube was less than 20% of total volume). Average enzyme velocity plotted over 120 hour time course (n=3).

h. Albumin-Fluorescein Isothiocyanate Conjugate (FITC-BSA)

The concentration was determined fluorometrically by measuring emission at λ=495 nm. Samples were placed in 10 ml of PBS and 200 μl of buffer was used to run the assay and not replaced (total amount withdrawn from tube was less than 40% of total volume). Average concentration plotted over 360 hour time course (n=3).

i. Results

Fibers were electrospun containing doxycycline, doxycycline-cyclodextrin complex, HRP, and FITC-BSA using an emulsion technique. More than 90% of Doxycycline was released within 6 h. Cyclodextrin-complexed doxycycline released at half the rate as pure doxycycline. HRP was released in active form with measurable activity at 24 h. FITC-BSA showed a much longer release profile with protein still released at 350 h.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

1. A co-electrospun polymeric fiber comprising;
a. a first polymer comprising a first pharmaceutically active agent or biologically active agent, wherein the first pharmaceutically active agent or biologically active agent is capable of release from the first polymer at a first release rate when the first polymer is not co-electrospun; and
b. a second polymer comprising a second pharmaceutically active agent or biologically active agent, wherein the second pharmaceutically active agent or biologically active agent is capable of release from the second polymer at a second release rate when the second polymer is not co-electrospun,
wherein the first release rate is greater than the second release rate,
wherein the first pharmaceutically active agent or biologically active agent and the second pharmaceutically active agent or biologically active agent are released from the co-electrospun polymeric fibers at a combined release rate between the first release rate and the second release rate.

2. The composition of claim 1, wherein the first polymer and the second polymer are different.

3. The composition of claim 1, wherein the first pharmaceutically active agent or biologically active agent and the second pharmaceutically active agent or biologically active agent are the same.

4. The composition of claim 1, further comprising a third polymer comprising a third pharmaceutically active agent or biologically active agent, wherein the third pharmaceutically active agent or biologically active agent is capable of release from the third polymer at a third release rate when the third polymer is not co-electrospun.

5. A bandage comprising the co-electrospun polymeric fiber of claim 1.

6. An implantable article comprising the co-electrospun polymeric fiber of claim 1.

7. A process for preparing a polymeric fiber capable of delivering a pharmaceutically active agent or biologically active agent comprising the steps of:
a. providing a first polymer comprising a first pharmaceutically active agent or biologically active agent, wherein the first pharmaceutically active agent or biologically active agent is capable of release from the first polymer at a first release rate when the first polymer is not co-electrospun;
b. providing a second polymer comprising a second pharmaceutically active agent or biologically active agent, wherein the second pharmaceutically active agent or biologically active agent is capable of release from the second polymer at a second release rate when the second polymer is not co-electrospun; and
c. co-electrospinning the first polymer with the second polymer, thereby providing co-electrospun polymeric fibers,
wherein the first release rate is greater than the second release rate,
wherein the first pharmaceutically active agent or biologically active agent and the second pharmaceutically active agent or biologically active agent are capable of release from the co-electrospun polymeric fibers at a combined release rate between the first release rate and the second release rate.

8. The process of claim 7, further comprising the step of providing a third polymer comprising a third pharmaceutically active agent or biologically active agent, wherein the third pharmaceutically active agent or biologically active agent is capable of release from the third polymer at a third release rate when the third polymer is not co-electrospun, wherein the third polymer is co-electrospun with the first polymer and the second polymer.

9. The product of the process of claim 7.

10. A process for preparing a polymeric fiber capable of delivering a pharmaceutically active agent or biologically active agent comprising the steps of:
a. co-electrospinning a first polymer with a second polymer, thereby providing co-electrospun polymeric fibers, and
b. impregnating the electrospun polymeric fibers with a pharmaceutically active agent or a biologically active agent, wherein the pharmaceutically active agent or the biologically active agent is capable of release from the first polymer at a first release rate when the first polymer is not co-electrospun and capable of release from the second polymer at a second release rate when the second polymer is not co-electrospun;
wherein the first release rate is greater than the second release rate;
wherein the pharmaceutically active agent or the biologically active agent is capable of release from the co-electrospun polymeric fibers at a combined release rate between the first release rate and the second release rate.

11. The product of the process of claim 10.

12. A process of modulating delivery rate of a pharmaceutically active agent or biologically active agent comprising the steps of:
a. providing a first amount of a first polymer comprising a first pharmaceutically active agent or biologically active agent, wherein the first pharmaceutically active agent or biologically active agent is capable of release from the first polymer at a first release rate when the first polymer is not co-electrospun;
b. providing a second amount of a second polymer comprising a second pharmaceutically active agent or biologically active agent, wherein the second pharmaceutically active agent or biologically active agent is capable of release from the second polymer at a second release rate when the second polymer is not co-electrospun; and
c. co-electrospinning the first polymer with the second polymer, thereby providing a co-electrospun polymeric fiber,
wherein the first release rate is greater than the second release rate,
wherein the first amount and the second amount are selected to provide a combined release rate for the co-electrospun polymeric fiber that is between the first release rate and the second release rate.

13. The process of claim 12, wherein the first polymer and the second polymer are different.

14. The process of claim 12, wherein the first pharmaceutically active agent or biologically active agent and the second pharmaceutically active agent or biologically active agent are the same.

15. A process of modulating delivery rate of a pharmaceutically active agent or biologically active agent comprising the steps of:
a. co-electrospinning a first amount of a first polymer with a second amount of a second polymer, thereby providing co-electrospun polymeric fibers, and
b. impregnating the electrospun polymeric fibers with a pharmaceutically active agent or a biologically active agent, wherein the pharmaceutically active agent or the biologically active agent is capable of release from the first polymer at a first release rate when the first polymer is not co-electrospun and capable of release from the second polymer at a second release rate when the second polymer is not co-electrospun;
wherein the first release rate is greater than the second release rate;
wherein the first amount and the second amount are selected to provide a combined release rate for the co-electrospun polymeric fiber that is between the first release rate and the second release rate.

16. A process of delivering a pharmaceutically active agent or biologically active agent, the method comprising the steps of:
a. providing a co-electrospun polymeric fiber comprising:
i. a first polymer comprising a first pharmaceutically active agent or biologically active agent, wherein the first pharmaceutically active agent or biologically active agent is capable of release from the first polymer at a first release rate when the first polymer is not co-electrospun; and
ii. a second polymer comprising a second pharmaceutically active agent or biologically active agent, wherein the second pharmaceutically active agent or biologically active agent is capable of release from the second polymer at a second release rate when the second polymer is not co-electrospun,
wherein the first release rate is greater than the second release rate,
wherein the first pharmaceutically active agent or biologically active agent and the second pharmaceutically active agent or biologically active agent are released from the co-electrospun polymeric fibers at a combined release rate between the first release rate and the second release rate; and
b. contacting the co-electrospun polymeric fiber with a subject, thereby delivering the first pharmaceutically active agent or biologically active agent and the second pharmaceutically active agent or biologically active agent at a combined release rate.

17. The process of claim 16, wherein the providing step comprises co-spinning the first polymer and the second polymer from an emulsion.

18. The process of claim 16, wherein the contacting step is implantation or topical administration.

19. The process of claim 16, wherein the first polymer is biodegradable.

20. The process of claim 16, further comprising the step of removing the co-electrospun polymeric fiber from the subject.
